(19) United States
(12) Patent Application Publication
(10) Pub. No.: US 2024/0142438 A1
(43) Pub. Date: May 2, 2024

(54) MULTI-ORGAN MODEL

(71) Applicant: CELLARTGEN INC., Seoul (KR)

(72) Inventors: Seung Woo CHO, Seoul (KR); Baofang CUI, Seoul (KR); Su Kyeom KIM, Seoul (KR); Su Ran Kim, Seoul (KR); Jung Ho BAE, Seoul (KR)

(21) Appl. No.: 18/539,379

(22) Filed: Dec. 14, 2023

Related U.S. Application Data
(63) Continuation of application No. PCT/KR2022/008401, filed on Jun. 14, 2022.

(30) Foreign Application Priority Data
Jun. 15, 2021 (KR) 10-2021-0077660
Jun. 14, 2022 (KR) 10-2022-0071910

(51) Int. Cl. G01N 33/50; C12N 5/071; C12N 5/077

(52) U.S. Cl. CPC G01N 33/5082; C12N 5/0657; C12N 5/0677; C12N 5/0679; C12N 5/0697; G01N 33/5038

(57) ABSTRACT

The present disclosure relates to a multi-organ model, and the multi-organ model of the present disclosure is excellent in culturing each organoid due to linkage between organoids and properties of hydrogel and can more accurately reflect the interactions between organs and the microenvironment in vivo. Also, the multi-organ model is further subjected to free fatty acid treatment and thus can more accurately mimic the phenotypes of non-alcoholic fatty liver. Accordingly, it is possible to analyze the effects of candidate drugs on peripheral organs.

### TECHNICAL FIELD

The present disclosure relates to a multi-organ model.

### BACKGROUND

Non-alcoholic fatty liver (NAFL) disease, whose basic lesion is fatty liver, is a disease state which exhibits tissue damage of the hepatic parenchyma such as inflammation, necrosis, and fibrosis that are similar to those in alcoholic liver injury, even without a long history of alcohol intake. NAFLD is basically asymptomatic. During the course of progression of the disease state, the fatty liver leads to steatohepatitis, liver cirrhosis, and then to liver cancer. The steatohepatitis in NAFLD is called non-alcoholic steatohepatitis (NASH). In particular, in recent years, metabolic syndromes due to obesity or diabetes have become a social problem, and NASH is also regarded as one of the metabolic syndromes. As complications of NAFLD and NASH, lifestyle-related diseases such as obesity, diabetes, hyperlipidemia, and hypertension are observed, and their major clinical manifestations characteristically include increases in the blood alanine aminotransferase (ALT) and hyaluronic acid levels, and on the other hand, decreases in the blood albumin level. However, the pathogenic mechanisms of NAFLD and NASH still remain largely unclear, and there is no established effective therapeutic method or therapeutic drug therefor at present. This is partly due to the fact that, since development of NAFLD and NASH is based on lifestyle-related diseases of humans, animal models suitable for research of NAFLD and NASH have not been established yet.

Elucidation of the disease pathology of NAFLD and NASH, which may progress to fatal diseases such as liver cirrhosis and liver cancer, is indispensable for development of effective therapeutic methods and therapeutic drugs therefor, and the elucidation requires appropriate models for NAFLD and NASH.

Animal models for NASH have so far been reported (for example, Patent Document 1). However, non-human animal models for NAFLD have been hardly reported. Further, due to recent issues related to animal ethics, the need for developing an effective in vitro model as an alternative to animal models has increased.

Furthermore, the occurrence and progression of various diseases in a human body and the reaction of the human body when a drug is administered are phenomena not occurring in one organ, but usually caused by complex interactions between various organs in the human body.

For example, metabolic diseases such as obesity, diabetes, and hypertension are caused by various factors such as eating habits, exercise, and stress, and various organ tissues such as the intestine, liver, immune system, and adipose tissue are known to be involved in the process.

Although the development of a test model capable of implementing the interactions between these organs can be of great help in researching disease mechanisms and developing therapeutic drugs, it cannot be achieved by conventional test methods since culture conditions are different for cells of each organ.

Thus, the inventors of the present disclosure completed the present disclosure as a result of researching a multi-organ model capable of reflecting the microenvironment of interactions between various organs.

### DISCLOSURE OF THE INVENTION

### Problems to be Solved by the Invention

An aspect of the present disclosure is conceived to provide a multi-organ model, including: a liver organoid well; and an intestinal organoid well, a pancreas organoid well, and a cardiac organoid well, each of which is directly or indirectly connected to the liver organoid well by microchannels.

Another aspect of the present disclosure is conceived to provide a non-alcoholic fatty liver multi-organ model in which the liver organoid well of the multi-organ model is treated with free fatty acid.

Yet another aspect of the present disclosure is conceived to provide a method of fabricating a non-alcoholic fatty liver multi-organ model, including: fabricating the multi-organ model; and injecting culture medium containing free fatty acid into the liver organoid well.

Still another aspect of the present disclosure is conceived to provide a method of screening a therapeutic drug for non-alcoholic fatty liver disease, including: treating the non-alcoholic fatty liver multi-organ model with a candidate substance; and comparing a group treated with the candidate substance to a control group, and a method of evaluating drug metabolism and drug toxicity on peripheral organs.

### Means for Solving the Problem

An aspect of the present disclosure provides a multi-organ model, including: a liver organoid well; and an intestinal organoid well, a pancreas organoid well, and a cardiac organoid well, each of which is directly or indirectly connected to the liver organoid well by microchannels.

In an embodiment of the present disclosure, the intestinal organoid well, the pancreas organoid well, and the cardiac organoid well may not be directly connected to each other.

In an embodiment of the present disclosure, the microchannels may have a cross-sectional width of 10 μm to 30 μm and a height of 5 μm to 20 μm.

In an embodiment of the present disclosure, the liver organoid well may include: a hydrogel containing decellularized liver tissue-derived extracellular matrix (Liver Extracellular Matrix; LEM); and liver organoids.

In an embodiment of the present disclosure, the intestinal organoid well may include a hydrogel containing decellularized intestinal tissue-derived extracellular matrix and intestinal organoids, the pancreas organoid well may include a hydrogel containing decellularized pancreas tissue-derived extracellular matrix and pancreas organoids, and the cardiac organoid well may include a hydrogel containing decellularized heart tissue-derived extracellular matrix and cardiac organoids.

In an embodiment of the present disclosure, the liver organoids may be derived from mouse tissue, human induced pluripotent stem cell (hiPSC) or human liver tissue.

Another aspect of the present disclosure provides a non-alcoholic fatty liver multi-organ model in which the liver organoid well of the multi-organ model is treated with free fatty acid.

In an embodiment of the present disclosure, the free fatty acid may have a concentration ranging from 100 μM to 900 μM.

Yet another aspect of the present disclosure provides a method of fabricating a non-alcoholic fatty liver multi-organ model, including: fabricating the multi-organ model; and injecting culture medium containing free fatty acid into the liver organoid well.

Still another aspect of the present disclosure provides a method of screening a therapeutic drug for non-alcoholic fatty liver disease, including: treating the non-alcoholic fatty liver multi-organ model with a candidate substance; and comparing a group treated with the candidate substance to a control group.

Still another aspect of the present disclosure provides a method of providing information about drug metabolism of a non-alcoholic fatty liver therapeutic drug on peripheral organs, including: treating the non-alcoholic fatty liver multi-organ model with a candidate substance; and comparing a group treated with the candidate substance to a control group.

Still another aspect of the present disclosure provides a method of evaluating drug toxicity of a non-alcoholic fatty liver therapeutic drug, including: treating the non-alcoholic fatty liver multi-organ model with a candidate substance; and comparing a group treated with the candidate substance to a control group.

### Effects of the Invention

A multi-organ model of the present disclosure is excellent in culturing each organoid due to linkage between organoids and properties of hydrogel and can more accurately reflect the interactions between organs and the microenvironment in vivo. Also, the multi-organ model is further subjected to free fatty acid treatment and thus can more accurately mimic the phenotypes of non-alcoholic fatty liver.

Further, since a non-alcoholic fatty liver multi-organ model of the present disclosure mimics non-alcoholic fatty liver, it can be used for screening a therapeutic drug for non-alcoholic fatty liver disease.

### BRIEF DESCRIPTION OF THE DRAWINGS

FIG. 1 is a diagram schematically illustrating fabricating a microfluidic device for implementing a multi-organ non-alcoholic steatohepatitis model. FIG. 2A and FIG. 2B are diagrams schematically illustrating fabricating a microfluidic device and dimensions for implementing a multi-organ non-alcoholic steatohepatitis model.

FIG. 3A to FIG. 3C show the results of analyzing a device culture environment through simulation. FIG. 4A and FIG. 4B show the results of analyzing a device culture environment through simulation. FIG. 5A to FIG. 5C show the results of analyzing a device culture environment through simulation. FIG. 6A and FIG. 6B show the results of analyzing a device culture environment through simulation. FIG. 7A to FIG. 7C show the results of analyzing a device culture environment through simulation.

FIG. 8A and FIG. 8B show the results of tests for optimizing the dimensions of a multi-organ microfluidic device.

FIG. 9A to FIG. 9D show the results of tests for optimizing the dimensions of a multi-organ microfluidic device.

FIG. 10 compares culture medium components for each organoid.

FIG. 11A to FIG. 11E show the results of confirming the possibility of culturing multi-organoids in a multi-organ microfluidic device.

FIG. 12 shows the result of comparing non-alcoholic steatohepatitis multi-organ models.

FIG. 13 shows the result of analyzing the effect on peripheral organs in a multi-organ non-alcoholic steatohepatitis organoid model (cultured in Matrigel).

FIG. 14 shows the result of analyzing the effect on peripheral organs in a multi-organ non-alcoholic steatohepatitis organoid model (cultured in decellularized tissue-derived extracellular matrix for each organ).

FIG. 15 shows the result of analyzing the effect on peripheral organs in a multi-organ non-alcoholic steatohepatitis organoid model.

FIG. 16A to FIG. 16C show the results of analyzing therapeutic effect of a candidate drug on NASH in a multi-organ non-alcoholic steatohepatitis organoid model (liver organoid).

FIG. 17 shows the result of analyzing therapeutic effect of a candidate drug on NASH in a multi-organ non-alcoholic steatohepatitis organoid model (liver organoid).

FIG. 18A and FIG. 18B show the results of analyzing the effect of a candidate drug on a peripheral organ (intestinal organoid) in a multi-organ non-alcoholic steatohepatitis organoid model.

FIG. 19 shows the result of analyzing the effect of a candidate drug on a peripheral organ (intestinal organoid) in a multi-organ non-alcoholic steatohepatitis organoid model.

FIG. 20A to FIG. 20C show the results of analyzing the effect of a candidate drug on a peripheral organ (pancreas organoid) in a multi-organ non-alcoholic steatohepatitis organoid model.

FIG. 21A to FIG. 21C show the results of analyzing the effect of a candidate drug on a peripheral organ (cardiac organoid) in a multi-organ non-alcoholic steatohepatitis organoid model.

FIG. 22A and FIG. 22B show the effect of a candidate drug on peripheral organs in a human induced pluripotent stem cell (hiPSC)-derived multi-organ non-alcoholic steatohepatitis organoid model.

FIG. 23A and FIG. 23B show the effect of a candidate drug on peripheral organs in a human induced pluripotent stem cell (hiPSC)-derived multi-organ non-alcoholic steatohepatitis organoid model.

### BEST MODE FOR CARRYING OUT THE INVENTION

Hereafter, the present disclosure will be described in detail with reference to the accompanying drawings. However, it is to be noted that the present disclosure is not limited to examples described herein but can be embodied in various other ways. It is to be understood that the term “comprises or includes” and/or “comprising or including” used in the document means that one or more other components, steps, operation and/or existence or addition of elements are not excluded in addition to the described components, steps, operation and/or elements unless context dictates otherwise.

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to a person with ordinary skill in the art and are described in numerous standard texts and reference works.

An aspect of the present disclosure provides a composition for hydrogel, including a phenol derivative-modified, tissue-derived extracellular matrix.

Unless otherwise defined herein, all technical and scientific terms used herein have the same meaning as commonly understood by a person with ordinary skill in the art to which this disclosure belongs.

Various scientific dictionaries that include the terms included herein are well known and available to those in the art. Although any methods and materials similar or equivalent to those described herein find use in the practice or testing of the disclosure, some preferred methods and materials are described. It is to be understood that this disclosure is not limited to the particular methodology, protocols, and reagents described, as these may vary, depending upon the context in which they are used by a person with ordinary skill in the art. Hereinafter, the present disclosure will be described in more detail.

An aspect of the present disclosure provides a multi-organ model, including: a liver organoid well; and an intestinal organoid well, a pancreas organoid well, and a cardiac organoid well, each of which is directly or indirectly connected to the liver organoid well by microchannels.

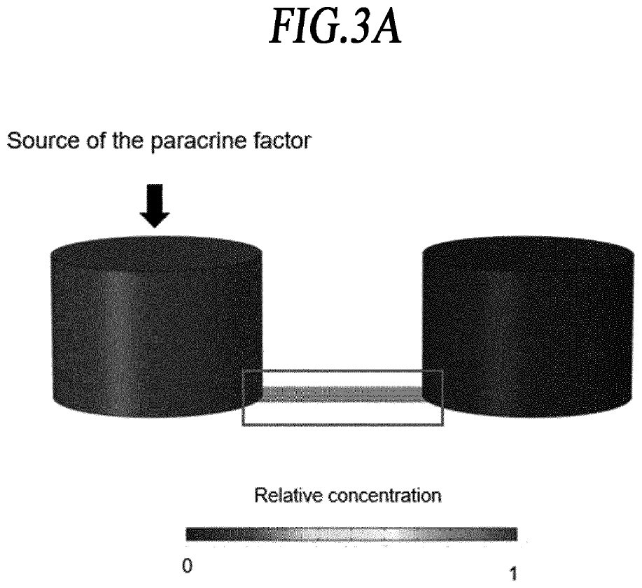

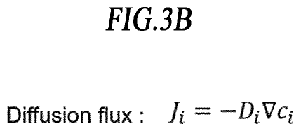

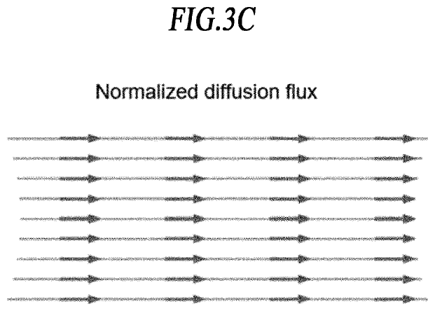

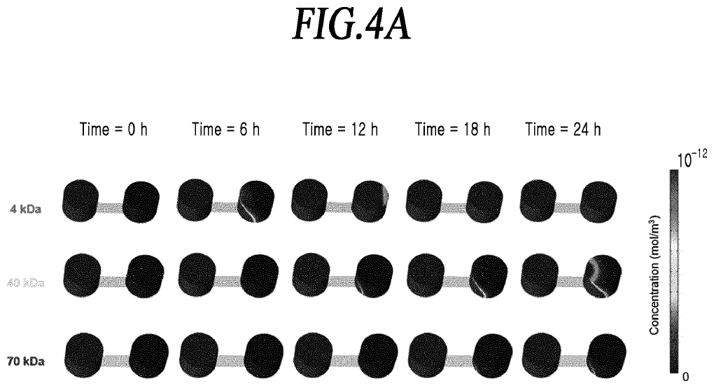

In an embodiment of the present disclosure, the intestinal organoid well, the pancreas organoid well, and the cardiac organoid well may not be directly connected to each other. In the past, attempts have been made to fabricate multi-organ models, but due to problems such as difficulty of fabrication/application, they were fabricated without considering the interactions between actual organs and the microenvironment. However, the multi-organ model of the present disclosure is composed of: the liver organoid well; the intestinal organoid well, the pancreas organoid well, and the cardiac organoid well, each of which is directly or indirectly connected to the liver organoid well by the microchannels, and is fabricated such that the intestinal organoid well, the pancreas organoid well, and the cardiac organoid well are not directly connected to each other. Thus, the multi-organ model of the present disclosure can more accurately reflect the interactions between organs in vivo, in particular, the in vivo microenvironment related to non-alcoholic fatty liver disease (see FIG. 2A to FIG. 3C).

In another embodiment of the present disclosure, the microchannels may have a cross-sectional width of 10 μm to 30 μm and a height of 5 μm to 20 μm, more specifically a width of 20 μm and a height of 10 μm. The dimensions of the microchannels are determined in consideration of the diffusion rate of paracrine factors in the microchannels, and the interactions between organs and the microenvironment may not be reflected in a microchannel having the sizes outside the above range. The number of microchannels between organoid wells may be one or more, and the microchannels may have a known shape and length.

In an embodiment of the present disclosure, the liver organoid well may include: a hydrogel containing decellularized liver tissue-derived extracellular matrix (Liver Extracellular Matrix; LEM); and liver organoids.

Also, in an embodiment of the present disclosure, the intestinal organoid well may include a hydrogel containing decellularized intestinal tissue-derived extracellular matrix and intestinal organoids, the pancreas organoid well may include a hydrogel containing decellularized pancreas tissue-derived extracellular matrix and pancreas organoids, and the cardiac organoid well may include a hydrogel containing decellularized heart tissue-derived extracellular matrix and cardiac organoids.

In an embodiment of the present disclosure, the LEM may be a matrix in which 95% to 99.9%, more specifically 96% to 98%, and most specifically 97.18% of liver tissue cells have been removed. Further, the decellularized intestinal tissue-derived extracellular matrix (Intestinal Extracellular Matrix; IEM) may be a matrix in which 95% to 99.9%, more specifically 96% to 98%, and most specifically 97.68% of intestinal tissue cells have been removed. Furthermore, the decellularized pancreas tissue-derived extracellular matrix (Pancreas Extracellular Matrix; PEM) may be a matrix in which 95% to 99.9%, more specifically 96% to 98%, and most specifically 96.03% of pancreas tissue cells have been removed. Also, the decellularized heart tissue-derived extracellular matrix (Heart Extracellular Matrix; HEM) may be a matrix in which 95% to 99.9%, more specifically 97% to 99%, and most specifically 98.72% of heart tissue cells have been removed. The organoids can be cultured in the respective tissue-derived extracellular matrices to better reflect the interactions between organs in vivo and the microenvironment specific to each organ.

The term “extracellular matrix” refers to a protein component found in mammals and multicellular organisms and a natural scaffold for cell growth that is prepared by decellularization of tissue. The extracellular matrix can be further processed through dialysis or crosslinking

The extracellular matrix may be a mixture of structural or non-structural biomolecules including, but not limited to, collagens, elastins, laminins, glycosaminoglycans, proteoglycans, antimicrobials, chemoattractants, cytokines and growth factors.

In mammals, the extracellular matrix may contain about 90% collagen in various forms. The extracellular matrices derived from various living tissues may differ in their overall structure and composition due to the unique role needed for each tissue.

The term “derive” or “derived” refers to a component obtained from any stated source by any useful method.

The term “organoid” refers to an ultraminiature body organ prepared in the form of an artificial organ by culturing cells derived from tissues or pluripotent stem cells in a 3D form.

The organoid is a three-dimensional tissue analog that contains organ-specific cells which originate from stem cells and self-organize (or self-pattern) in a similar manner to the in vivo condition. The organoid can be developed into a specific tissue by patterning restricted elements (for example, a growth factor).

The organoid can have the intrinsic physiological properties of the cells and can have an anatomical structure that mimics the original state of a cell mixture (multiple cell types including all remaining stem cells and the neighboring physiological niche). A three-dimensional culture method allows the organoid to be better arranged in terms of cell to cell functions and to have an organ-like form with functionality and tissue-specific functions.

In an embodiment of the present disclosure, the liver organoid may be derived from mouse tissue, human induced pluripotent stem cells (hiPSC) or human liver tissue, more specifically human induced pluripotent stem cells (hiPSC) or human liver tissue.

The multi-organ model may be prepared by fabricating a multi-organ model device, including: a liver organoid well; and an intestinal organoid well, a pancreas organoid well, and a cardiac organoid well, each of which is directly or indirectly connected to the liver organoid well by microchannels; and locating and culturing each organ-derived extracellular matrix and tissue cells in each organoid well.

Another aspect of the present disclosure provides a non-alcoholic fatty liver multi-organ model in which the liver organoid well of the multi-organ model is treated with free fatty acid.

The free fatty acid treatment may be performed directly onto a liver organoid well including the cultured liver organoids, or may be performed simultaneously with liver organoid culture or after a liver organoids are cultured and then mixed with culture medium.

In an embodiment of the present disclosure, the free fatty acid may have a concentration ranging from 100 μM to 900 μM, specifically 200 μM to 800 μM, and most specifically 500 μM. The liver organoid well is treated with the free fatty acid, which makes the liver organoids exhibit the characteristics of non-alcoholic fatty liver, and factors secreted by the non-alcoholic fatty liver organoids flow through the microchannels to the intestinal organoid well, the pancreas organoid well, and the cardiac organoid well and thus affect each organoid. If the free fatty acid is used at a concentration out of the above range, the characteristics of non-alcoholic fatty liver may not appear or the cells in the model may die.

Also, if the free fatty acid treatment is performed simultaneously with liver organoid culture or after liver organoids are cultured and then mixed with culture medium, the components of the culture medium may be a mixture of known substances used for liver organoid culture in addition to free fatty acids.

In an embodiment of the present disclosure, the free fatty acid may be any one selected from oleic acid, palmitic acid and linoleic acid, and may be specifically oleic acid.

Yet another aspect of the present disclosure provides a method of fabricating a non-alcoholic fatty liver multi-organ model, including: fabricating the multi-organ model; and injecting culture medium containing free fatty acid into the liver organoid well.

The fabricating the multi-organ model is to fabricate the multi-organ model. Specifically, it may be composed of fabricating a device (microfluidic chip) including each organoid chamber and microchannels by using PDMS polymer; and locating the tissue-derived extracellular matrices and tissue cells in the respective wells. Details of the extracellular matrix and organoid are the same as those of the non-alcoholic fatty liver artificial tissue model described above.

The injecting is to inject the culture medium containing the free fatty acid into the multi-organ model. As described above, the culture medium containing the free fatty acid flows into the well in which the liver organoid is located, and the liver organoids are exposed to the free fatty acid and thus exhibit the phenotypes of non-alcoholic fatty liver.

Still another aspect of the present disclosure provides a method of screening a therapeutic drug for non-alcoholic fatty liver disease, including: treating the non-alcoholic fatty liver multi-organ model with a candidate substance; and comparing a group treated with the candidate substance to a control group.

The treating with the candidate substance is to treat the non-alcoholic fatty liver multi-organ model with the candidate substance, and the treatment with the candidate substance may vary depending on the intended route of administration and dosage of the candidate substance.

Further, the comparing the group treated with the candidate substance to the control group may be to compare the non-alcoholic fatty liver multi-organ model treated with the candidate substance to the control group. The control group may be a non-alcoholic fatty liver multi-organ model treated with or not treated with a conventionally known non-alcoholic fatty liver therapeutic drug or a known substance within the range that does not inhibit or increase the physiological activity of the liver organoid in the non-alcoholic fatty liver multi-organ model.

The group treated with the candidate substance may be compared to the control group by analyzing the levels of fat accumulation in the liver organoids, the differentiation and functionality of the liver organoids, the levels of inflammation and fibrosis, and the viability of the liver organoids and/or by checking various indicators in the liver organoids or culture media.

The method of screening may further include selecting a non-alcoholic fatty liver therapeutic drug. The selecting may be to select a non-alcoholic fatty liver therapeutic drug when a reduction of fat accumulation in the liver organoids, the recovery of differentiation and functionality of the liver organoids, a decrease in levels of inflammation and fibrosis, an increase in viability of the liver organoids, and/or an increase in improved indicators in the liver organoids or culture media are confirmed in the above-described process of comparing. If a conventionally known non-alcoholic fatty liver therapeutic drug is used as a control group, any non-alcoholic fatty liver therapeutic drug can be determined and selected as having an improved effect compared to the conventionally known non-alcoholic fatty liver therapeutic drug when it shows an improved effect compared to the control group. Also, the multi-organ model of the present disclosure includes organoids of major human organs, i.e. the liver, intestine, pancreas, and heart and reflects their microenvironment. Thus, it is possible to not only select a non-alcoholic fatty liver therapeutic drug but also check the effect of the selected therapeutic drug on other organs. Further, it is possible to select a drug capable of reducing, or improving stress applied to other organs in the environment of non-alcoholic fatty liver.

Still another aspect of the present disclosure provides a method of providing information about drug metabolism of a non-alcoholic fatty liver therapeutic drug on peripheral organs, including: treating the non-alcoholic fatty liver multi-organ model with a candidate substance; and comparing a group treated with the candidate substance to a control group, and a method of evaluating drug toxicity of the non-alcoholic fatty liver therapeutic drug.

The treating and the comparing are the same as described above.

Meanwhile, the method may further include evaluating drug metabolism of a non-alcoholic fatty liver therapeutic drug on peripheral organs; or evaluating drug toxicity of the non-alcoholic fatty liver therapeutic drug. The selecting may be to evaluate the method of providing information about drug metabolism of a therapeutic drug on peripheral organs and drug toxicity when a reduction of fat accumulation in the liver organoids, the recovery of differentiation and functionality of the liver organoids, a decrease in levels of inflammation and fibrosis, an increase in viability of the liver organoids, and/or an increase in improved indicators in the liver organoids or culture media are confirmed in the above-described process of comparing. If a conventionally known non-alcoholic fatty liver therapeutic drug is used as a control group, any non-alcoholic fatty liver therapeutic drug can be determined and selected as having an improved effect compared to the conventionally known non-alcoholic fatty liver therapeutic drug when it shows an improved effect compared to the control group.

### MODE FOR CARRYING OUT THE INVENTION

Hereinafter, one or more specific embodiments will be described in more detail through examples. However, these examples are for illustrative purposes of one or more embodiments, and the scope of the present disclosure is not limited to these examples.

### Example 1: Fabrication of Microfluidic Device for Implementing Multi-Organ Non-Alcoholic Steatohepatitis Model

To implement a multi-organ non-alcoholic steatohepatitis model, a microdevice in which liver, intestinal, pancreas and cardiac organoids can be organically connected and co-cultured was fabricated.

In actual non-alcoholic steatohepatitis (NASH) patients, various organs are affected by fatty liver with excessive fat accumulation. Particularly, it is known that the intestine, heart, and pancreas are affected. Actually, NASH patients may often develop an inflammatory bowel disease in the small intestine or have leaky gut due to damage to the wall of the small intestine. Also, NASH patients may develop cardiovascular disease and arrhythmia in the heart. Further, NASH patients may develop acute pancreatitis in the pancreas or have a decrease in function of insulin-secreting beta cells. Therefore, it is very important to consider the effects on other organs together in the development of non-alcoholic steatohepatitis drugs and the treatment of actual patients.

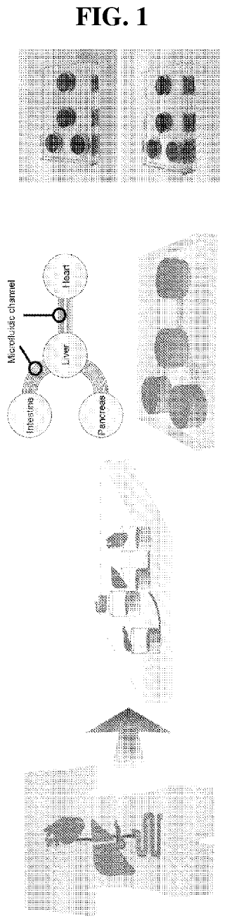

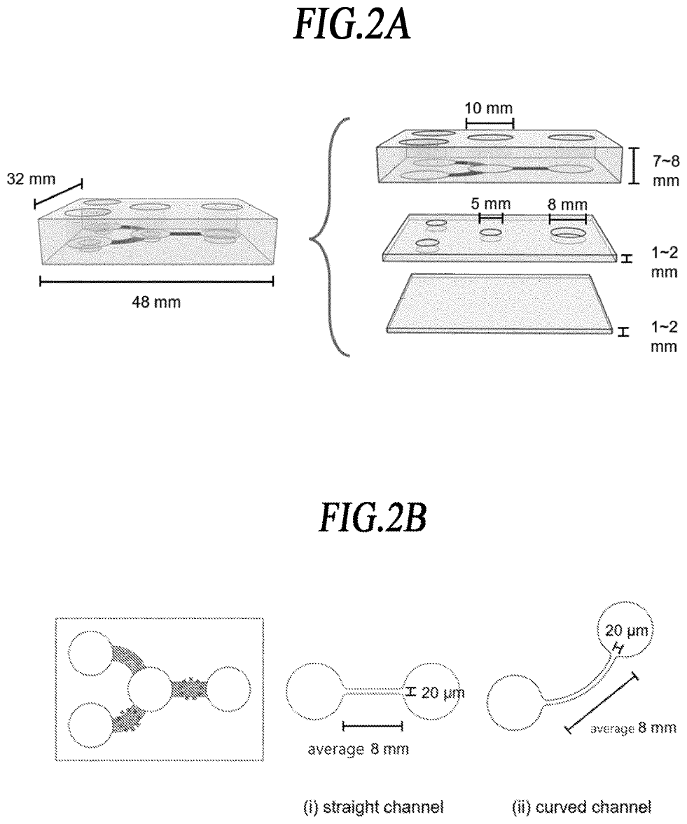

To this end, a multi-organ NASH co-culture model, which cannot be implemented in a conventional well-plate, was implemented by using a microfluidic device. The organoids were respectively cultured in divided compartments in one device, and each organoid was cultured in the culture medium suitable for the corresponding organoid. Also, the organoids were connected to mimic the locations, sequence, and metabolic process of the respective organs in the body, and could interact with each other through microfluidic channels (FIG. 1).

Specifically, the microfluidic device had a horizontal length of 48 mm and a vertical length of 32 mm, and was fabricated to have a total of three layers. The lowermost layer is a non-patterned bottom layer, and has a thickness of 1 mm to 2 mm. The intermediate layer is a patterned layer for seeding organoids, and has a thickness of 1 mm to 2 mm and a pattern diameter of 5 mm or 8 mm. The uppermost layer is a layer including microfluidic channels and organoid culture chambers, and has a thickness of 7 to 8 mm and an organoid culture chamber diameter of 10 mm Basically, the microfluidic device can be fabricated using a polydimethylsiloxane (PDMS) polymer by soft lithography (FIG. 2A).

There are two types of microfluidic channels: (i) straight channel and (ii) curved channel. The both channels have a width of 20 μm, an average length of 8 mm, and a height of 10 μm. The organoid chambers were connected through a total of 9 microfluidic channels (FIG. 2B).

### Test Example 1: Analysis of Culture Environment Through Simulation

When non-alcoholic steatohepatitis occurs, various harmful substances such as inflammatory cytokines are produced and these harmful substances affect the peripheral organs. Therefore, it is essentially required to check the effects of these harmful substances on other organs through a multi-organ non-alcoholic steatohepatitis model. Simulation analysis was conducted to check the transfer of these harmful substances in the fabricated microfluidic device.

In the microfluidic device, the substances are transferred between the organoid chambers mainly by means of diffusion (FIG. 3A). A paracrine factor generated in each organoid chamber diffuses through the microfluidic channels and affects the organoids in the peripheral chambers. The diffusion flux of the paracrine factor is represented by J_i=D_i∇c_i according to Fick's law of diffusion. J_i denotes the diffusion flux of a paracrine factor i, D_i denotes the diffusion coefficient of the paracrine factor i, and ∇c_i denotes the concentration gradient of the paracrine factor i (FIG. 3B). FIG. 3C is an enlarged view of the portion indicated by the red rectangular frame in FIG. 3A, and shows the diffusion flux when the paracrine factor diffuses through the channels.

The organoid culture medium contains various growth factors, and in the non-alcoholic steatohepatitis model, cells at a disease site secrete various inflammatory cytokines. Simulation analysis was conducted to predict the transfer of these growth factors and paracrine factors in the microfluidic device.

To simulate concentration changes of growth factors and paracrine factors that diffuse over time, simulation analysis was conducted for 24 hours on concentration changes caused by diffusion, through microfluidic channels, of FITC-dextran of 4 kDa, 40 kDa and 70 kDa corresponding to a molecular weight range of growth factors and paracrine factors present in the body. FITC-dextran of each molecular weight was set to be present in one side culture chamber (left chamber) of the device at a concentration of 0.01 mol/m³ at Time=0 h. FITC-dextran of 4 kDa has a diffusion coefficient of 1.35×10⁻¹⁰ m²/s in the culture medium, FITC-dextran of 40 kDa has a diffusion coefficient of 4.5×10⁻¹¹ m²/s in the culture medium, and FITC-dextran of 70 kDa has a diffusion coefficient of 2.3×10⁻¹¹ m²/s in the culture medium. At Time=6 h, the concentration of FITC-dextran of 4 kDa was first checked in the opposite side (right) culture chamber, and at Time=12 h, the concentration of FITC-dextran of 40 kDa was checked in the opposite side (right) culture chamber, and at Time=18 h, the concentration of FITC-dextran of 70 kDa was finally checked in the opposite side (right) culture chamber (FIG. 4A).

Quantitative analysis was conducted on diffusion patterns of FITC-dextran of 4 kDa, 40 kDa and 70 kDa in the microfluidic channel for 24 hours. In FIG. 4B, FITC-dextran of 4 kDa is shown in red, FITC-dextran of 40 kDa is shown in green, and FITC-dextran of 70 kDa is shown in blue. The concentration measurement point for FITC-dextran of each molecular weight is indicated by a dot with a color corresponding to FITC-dextran of each molecular weight in the device diagram on the left of FIG. 4B. It was observed that the concentrations of FITC-dextran of 4 kDa, 40 kDa and 70 kDa increased over time at the points indicated by the dots, respectively (right graph in FIG. 4B). FITC-dextran of each molecular weight had a concentration of 0.01 mol/m³ at Time=0 h. At Time=24 h, FITC-dextran of 4 kDa, FITC-dextran of 40 kDa, and FITC-dextran of 70 kDa had a concentration of 7.6431×10⁻⁶ mol/m³, 9.0836×10⁻⁷ mol/m³, and 3.5724×10⁻⁸ mol/m³, respectively, at the marked points in the opposite site culture chamber.

Further, to predict material transfer in the microfluidic device applied to the multi-organ non-alcoholic steatohepatitis model, simulation analysis was conducted on a diffusion pattern of 40 kDa FITC-dextran from in an intermediate culture chamber (chamber where liver organoids were cultured) to peripheral culture chambers over time.

As a result, as shown in FIG. 5A, it was confirmed that 40 kDa FITC-dextran began to diffuse from the intermediate chamber at Time=0 h and diffused into the peripheral chambers at Time=24 h.

FIG. 5B is an enlarged view of the portion indicated by the red rectangular frame at Time=1 h in FIG. 5A, and shows that 40 kDa FITC-dextran is diffusing through the channel and is not yet sufficiently diffused into the peripheral culture chambers at Time=1 h.

FIG. 5C is an enlarged view of the portion indicated by the red rectangular frame at Time=3 h in FIG. 5A, and shows that 40 kDa FITC-dextran is further diffused and reaches the peripheral culture chambers at Time=3 h.

Furthermore, to predict material transfer in the microfluidic device applied to the multi-organ non-alcoholic steatohepatitis model, diffusion of 40 kDa FITC-dextran from the intermediate culture chamber (chamber where liver organoids were cultured) to peripheral culture chambers for 24 hours was simulated, followed by quantitative analysis.

Specifically, the concentration of 40 kDa FITC-dextran diffused through the microfluidic channel for 24 hours was checked at the point indicated by the red dot in the device diagram on the left of FIG. 6A. As a result, as shown in FIG. 6A, it was confirmed that at Time=0 h, the start time of the simulation, the concentration of 40 kDa FITC-dextran at the point indicated by the red dot was 0.0000 mol/m³, and at Time=24 h, the end time of the simulation, the concentration of 40 kDa FITC-dextran at the point indicated by the red dot was 8.1388×10⁻⁷ mol/m³.

Further, the concentration of 40 kDa FITC-dextran diffused through the microfluidic channel at Time=24 h was analyzed. It was confirmed that at Time=24 h, the concentration at Distance=0 mm, the left end point of the microfluidic channel in the red line in the device diagram of FIG. 6 B, was 0.0099769 mol/m³ and the concentration at Distance=8 mm, the right end point of the microfluidic channel, was 8.1388×10⁻⁷ mol/m³.

Furthermore, to identify diffusion patterns of the paracrine factors secreted from the peripheral culture chambers of the microfluidic device applied to the multi-organ non-alcoholic steatohepatitis model to the intermediate culture chamber (chamber where liver organoids were cultured), diffusion of 40 kDa FITC-dextran having a concentration of 0.01 mol/m³ at Time=0 for 24 hours was simulated and analyzed.

As a result, it was confirmed that 40 kDa FITC-dextran secreted from the culture chamber located at the top left was diffused into the intermediate culture chamber at Time=24 h (FIG. 7A). Also, it was confirmed that 40 kDa FITC-dextran secreted from the culture chamber located at the bottom left was diffused into the intermediate culture chamber at Time=24 h (FIG. 7B). Besides, it was confirmed that 40 kDa FITC-dextran secreted from the culture chamber located on the right was diffused into the intermediate culture chamber at Time=24 h (FIG. 7C).

As a result of analyzing this series of simulations, it was confirmed that various growth factors and paracrine factors, such as inflammatory cytokines, secreted by cells in the non-alcoholic steatohepatitis model can be diffused through the microfluidic channel. Therefore, it was verified that the fabricated microfluidic device can be used to evaluate interactions between organs and drug metabolism/toxicity in multi-organ non-alcoholic steatohepatitis modeling.

### Test Example 2: Optimization of Dimensions of Multi-Organ Microfluidic Device

In order to construct a multi-organ non-alcoholic steatohepatitis model, growth factors and paracrine factors need to be diffused at an optimal rate through a channel connecting organoid chambers. In the microfluidic device, the diffusion rates of growth factors and paracrine factors are determined by channel dimensions (height, width, length, and number of channels). When a multi-organoid-based non-alcoholic steatohepatitis (NASH) model is constructed, four different types of organoids are affected by paracrine factors, such as inflammatory cytokines, secreted from non-alcoholic steatohepatitis-induced organoids. However, growth factors required for culture and included in each organoid culture chamber need to be maintained at certain levels in order to normally culture the four different types of organoids. To this end, organoids were cultured in a device equipped with diffusion channels of various dimensions and an optimal device for a multi-organ model was selected. Liver and pancreas organoids were obtained by extracting adult stem cells from a mouse tissue, encapsulated in a decellularized liver tissue-derived matrix (6 mg/ml) and a decellularized pancreas tissue-derived matrix (4 mg/ml), respectively, at a cell density of 70,000 cells/30 μL gel, and cultured. An intestinal organoid was obtained by extracting intestinal crypts from a mouse intestinal tissue and encapsulated in a decellularized intestinal tissue-derived matrix (2 mg/ml) at a cell density of 800 crypts/30 μL gel, and cultured. A cardiac organoid composed of cardiomyocytes was prepared by three-dimensionally culturing mouse embryonic fibroblasts in microwells, followed by direct reprogramming chemically induced by the culture medium components. The cardiac organoid was cultured on a hydrogel (20 organoids/70 μL gel bed) prepared by crosslinking of a decellularized cardiac tissue-derived matrix (5 mg/ml) in the form of a 70 μL gel bed at the bottom of the device. The decellularized matrix for culturing each organoid was applied at the optimal concentration for differentiation of each organoid as determined through previous research.

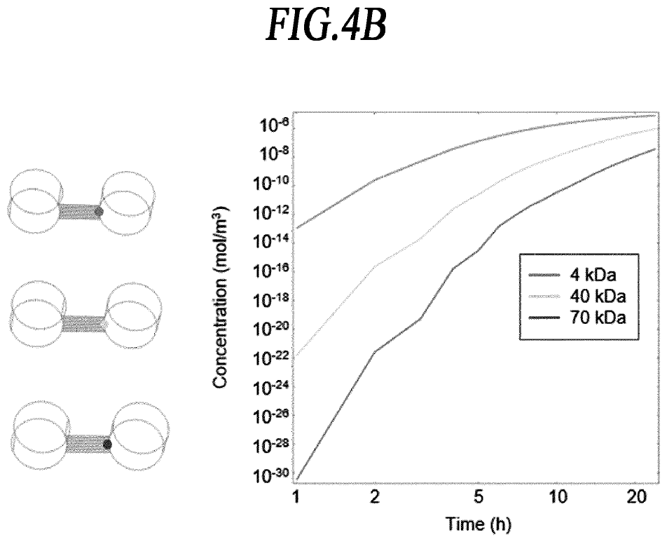

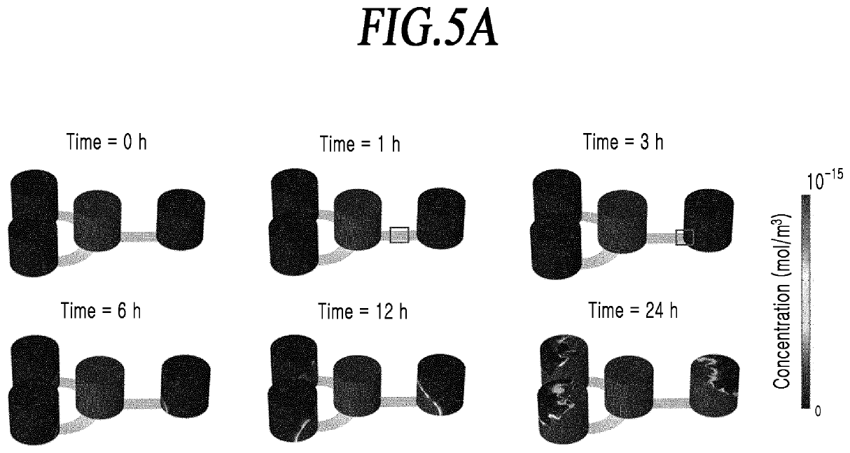

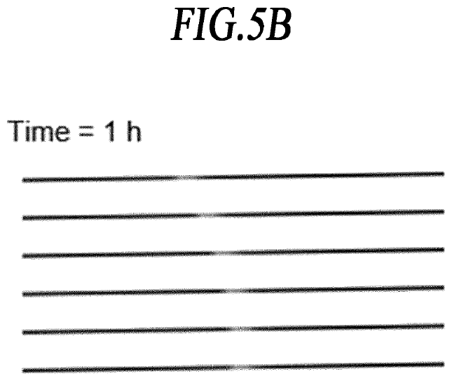

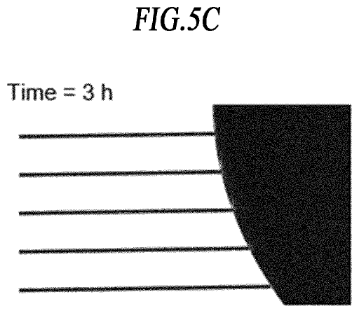

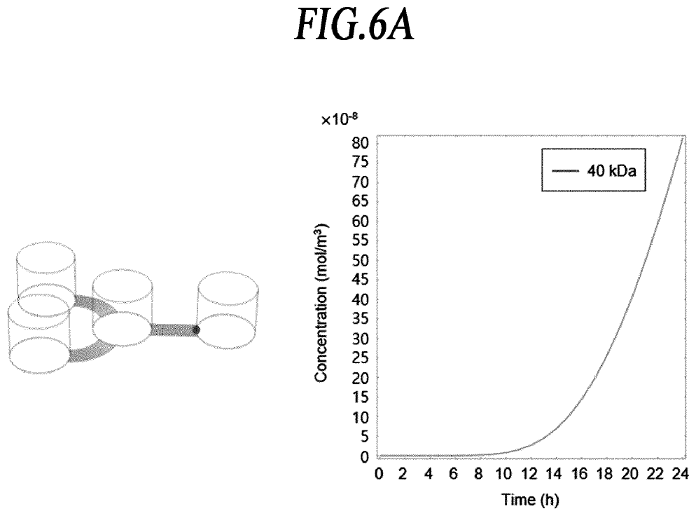

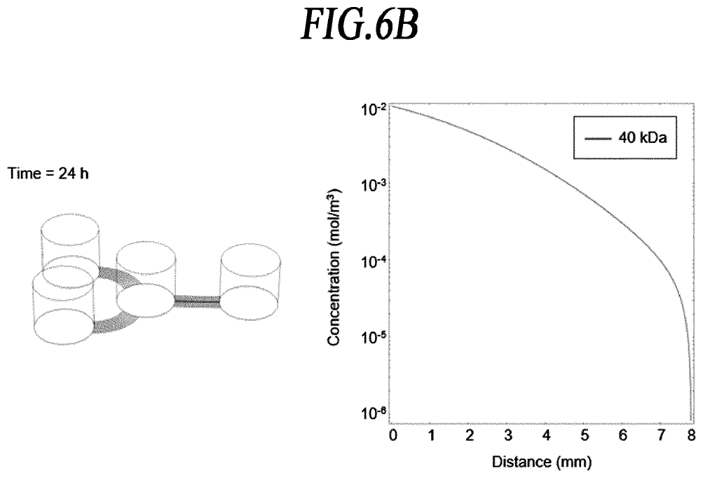

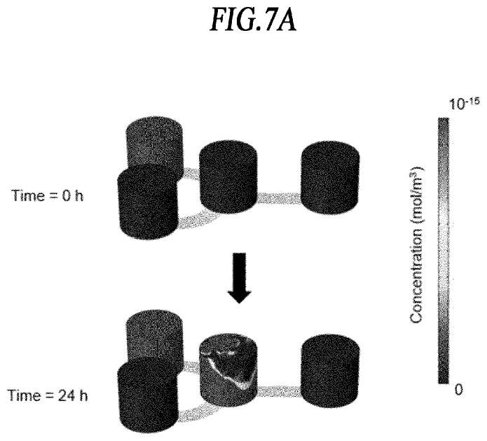

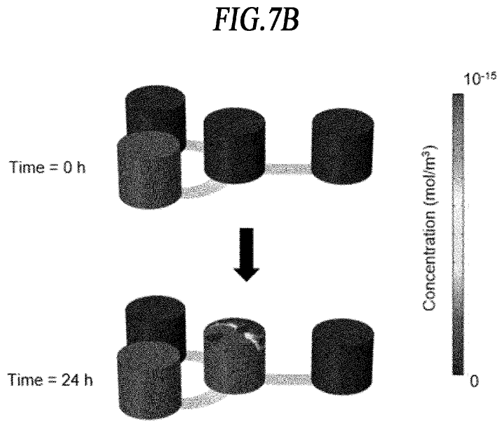

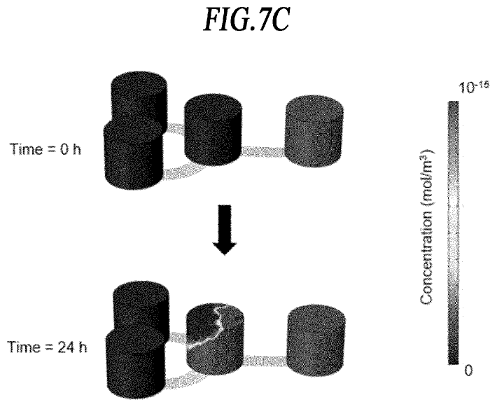

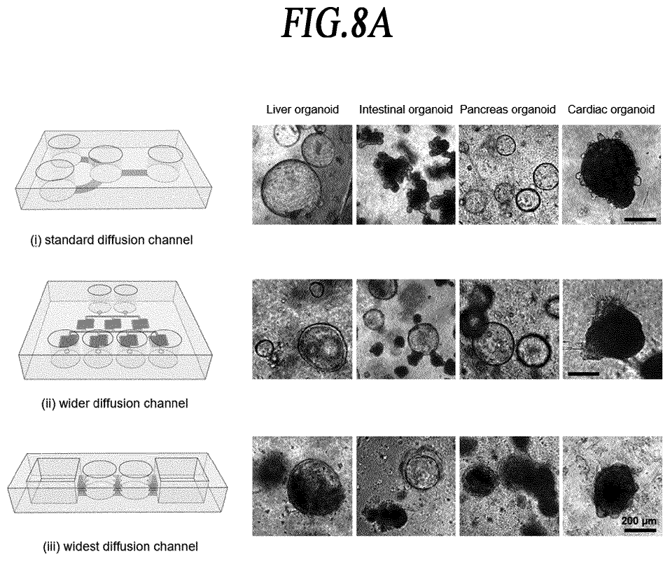

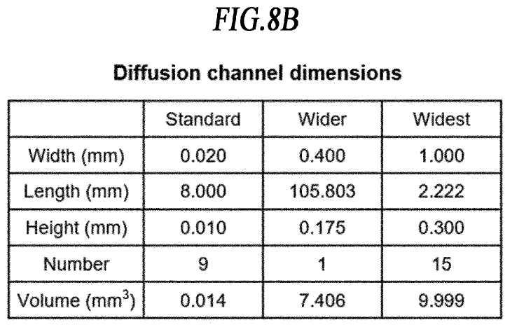

Three devices with different channel dimensions shown in FIG. 8A were used to check whether diffusion of growth factors and paracrine factors in each device is suitable for multi-organoid culture and non-alcoholic steatohepatitis model construction. When liver, intestinal, pancreas and cardiac organoids were cultured for 3 days by using each device, it was confirmed that four different types of organoids were normally developed and appropriately cultured in the standard diffusion channel (i). Meanwhile, the liver organoid cultured in the wider diffusion channel (ii) was smaller in size and grew slower than the liver organoid cultured in the standard diffusion channel, and an intestinal organoid in a compact form, which is conventionally normal, was observed in addition to an intestinal organoid in a cystic form. Also, protrusion of some of the cardiomyocytes from the inside to the outside of the cardiac organoid was observed, which means that the organoid was structurally deformed. It seems that diffusion of the culture medium between the organoid culture chambers occurred to some extent in the wider diffusion channel compared to the standard diffusion channel, and, thus, the culture medium components and growth factors specific to each organ were partially mixed in the other organoid culture media, which made it difficult to completely culture each organoid. It seems that in the widest diffusion channel (iii), four different types of organoid culture media were mixed with each other, and, thus, liver, intestinal and pancreas organoids were not appropriately generated and developed and a cardiac organoid was significantly reduced in size, which confirmed that the device with the widest diffusion channel dimensions is not suitable for co-culture of multi-organoids.

The device with the standard diffusion channel dimensions (i) had a channel width of 0.020 mm, a channel length of 8.000 mm, and a channel height of 0.010 mm, and in the device, the organoid culture chambers were connected by nine channels. Further, the total volume of the channels was 0.014 mm³. The device with the wider diffusion channel dimensions (ii) had a channel width of 0.400 mm, a channel length of 105.803 mm, and a channel height of 0.175 mm, and in the device, the organoid culture chambers were connected by one channel. Further, the total volume of the channel was 7.406 mm³. The device with the widest diffusion channel dimensions (iii) had a channel width of 1.000 mm, a channel length of 2.222 mm, and a channel height of 0.300 mm, and in the device, the organoid culture chambers were connected by 15 channels. Further, the total volume of the channels was 9.999 mm³ (FIG. 8B).

### Test Example 3: Optimization of Dimension of Multi-Organ Microfluidic Device

In order to construct a multi-organ non-alcoholic steatohepatitis model, growth factors and paracrine factors need to be diffused at an optimal rate through a channel connecting organoid chambers. In the microfluidic device, the diffusion rates of growth factors and paracrine factors are determined by channel dimensions (height, width, length, and number of channels). When a multi-organoid-based non-alcoholic steatohepatitis (NASH) model is constructed, four different types of organoids are affected by paracrine factors, such as inflammatory cytokines, secreted from non-alcoholic steatohepatitis-induced organoids. However, growth factors required for culture and included in each organoid culture chamber need to be maintained at certain levels in order to normally culture the four different types of organoids. To this end, organoids were cultured in a device equipped with diffusion channels of various dimensions and an optimal device for a multi-organ model was selected. Liver and pancreas organoids were obtained by extracting adult stem cells from a mouse tissue, encapsulated in a decellularized liver tissue-derived matrix (6 mg/ml) and a decellularized pancreas tissue-derived matrix (4 mg/ml), respectively, at a cell density of 70,000 cells/30 μL gel, and cultured. An intestinal organoid was obtained by extracting intestinal crypts from a mouse intestinal tissue and encapsulated in a decellularized intestinal tissue-derived matrix (2 mg/ml) at a cell density of 800 crypts/30 μL gel, and cultured. A cardiac organoid composed of cardiomyocytes was prepared by three-dimensionally culturing mouse embryonic fibroblasts in microwells, followed by direct reprogramming chemically induced by the culture medium components. The cardiac organoid was cultured on a hydrogel (20 organoids/70 μL gel bed) prepared by crosslinking of a decellularized cardiac tissue-derived matrix (5 mg/ml) in the form of a 70 μL gel bed at the bottom of the device. The decellularized matrix for culturing each organoid was applied at the optimal concentration for differentiation of each organoid as determined through previous research.

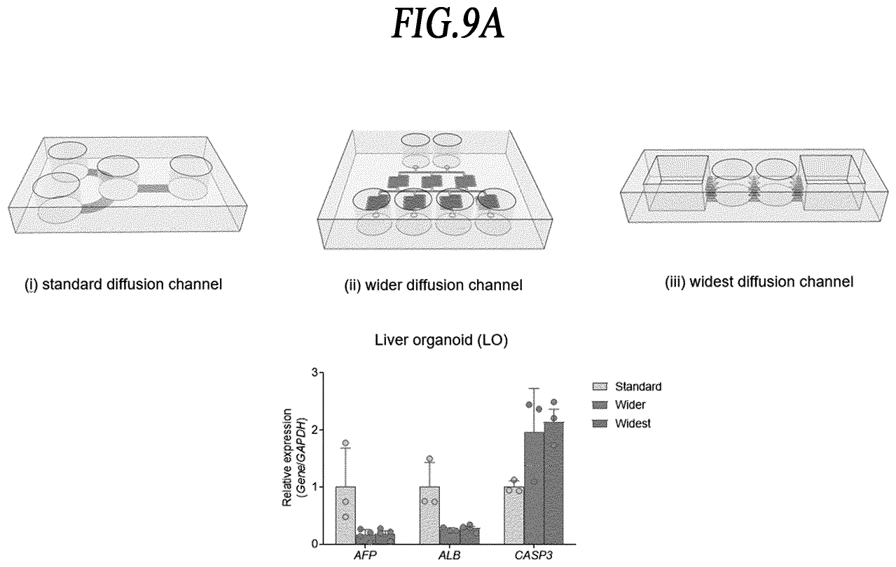

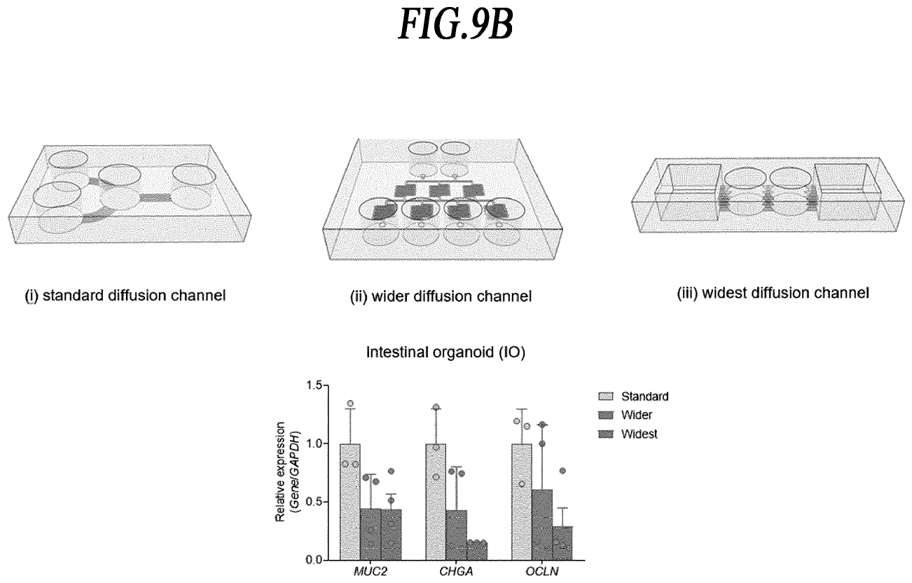

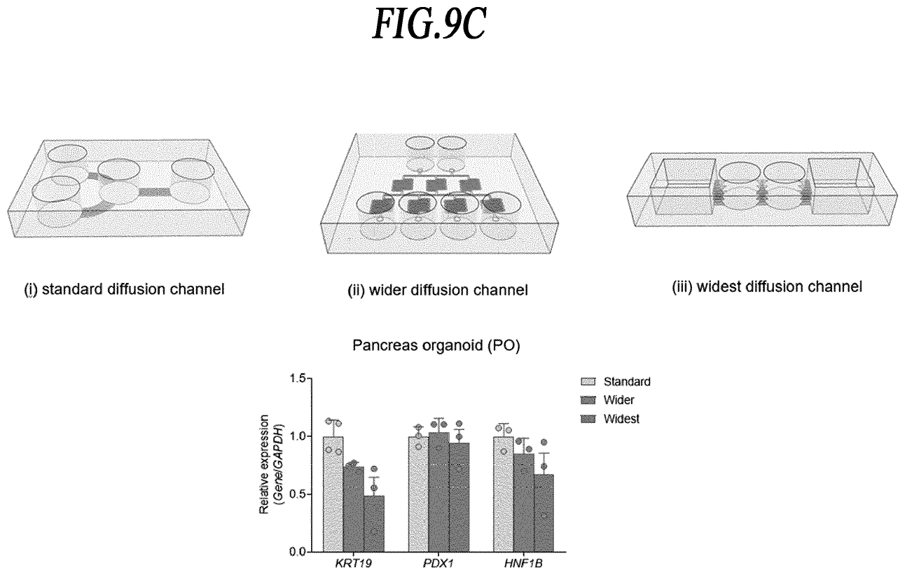

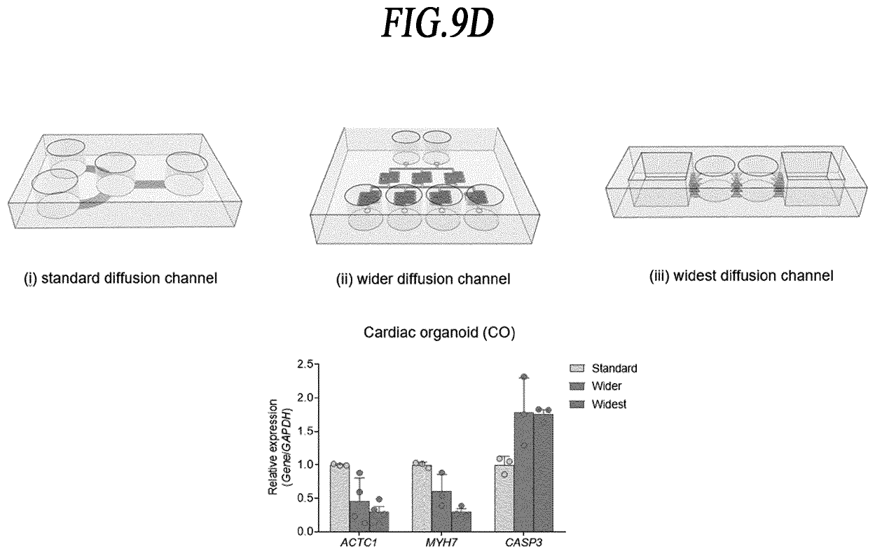

Multi-organoids were cultured for 3 days in three types of devices with different diffusion channel dimensions and chamber arrangement, followed by quantitative PCR analysis to compare the expression of each organ-specific differentiation marker gene. It was confirmed that the liver organoid cultured in the standard diffusion channel (i) showed the highest expression levels of hepatic differentiation markers, AFP and ALB, and the liver organoids cultured in the wider diffusion channel (ii) and the widest diffusion channel (iii) showed remarkably low expression levels of the differentiation markers. Also, it was confirmed that CASP3, an apoptosis marker, had the lowest expression level in a multi-organ chip with the standard channel design (i) selected as the optimal device in the present development, and the expression level of the apoptosis marker increased in the order of the wider channel device (ii) and the widest channel device (iii) (FIG. 9A).

As for intestinal organoids, it was confirmed that the expression levels of intestinal differentiation markers, MUC2 and CHGA, and a gut barrier marker, OCLN, were the highest in the multi-organ chip with the standard channel (i), and gradually decreased in the order of the wider channel device (ii) and the widest channel device (iii) (FIG. 9B).

As for pancreas organoids, it was confirmed that there was no significant difference in the expression of PDX1, a pancreatic endoderm marker, among the three types of devices, but the expression levels of KRT19 and HNF1B were the highest in the standard channel multi-organ device (i) selected as the optimal design in the present disclosure, and decreased in the order of the wider channel device (ii) and the widest channel device (iii) (FIG. 9C).

As a result of comparing the gene expression of cardiac organoids cultured in the three types of devices, it was confirmed that the expression levels of cardiac differentiation markers, ACTC1 and MYH7, were the highest in the standard channel device (i), and decreased in the order of the wider channel device (ii) and the widest channel device (iii). On the contrary, the expression level of CASP3, an apoptosis marker, was the lowest in the standard channel device (i), and increased as the size of the channel increased (FIG. 9D).

Accordingly, it was confirmed that the device with the standard diffusion channel dimensions and design (i) is capable of performing co-culture while maintaining high differentiation potency of each of the four different types of multi-organoids (liver, intestine, pancreas, and heart) and minimizing apoptosis. Therefore, it was used later as a device for multi-organ culture.

### Test Example 4: Comparison of Each Culture Medium Component of Multi-Organoid

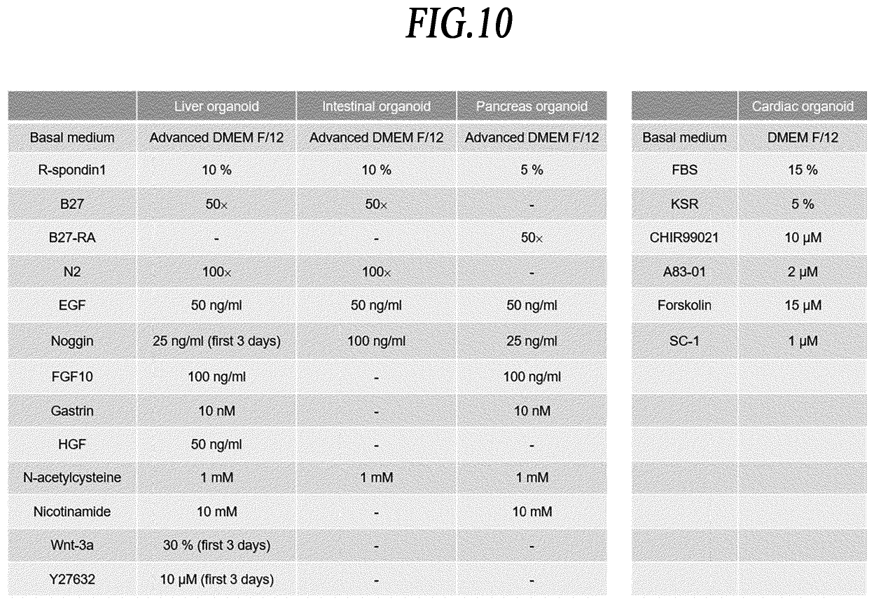

Liver, intestinal and pancreas organoids were cultured by using the standard culture medium components, which have been most widely used in culture media for mouse adult stem cell-derived organoids, and the composition thereof is shown in FIG. 10.

A cardiac organoid was cultured by using the composition of a representative culture medium used in a chemically induced protocol for direct reprogramming from mouse embryonic fibroblasts to cardiomyocytes. Since each organoid has different culture medium composition and growth factor concentration optimized for growth and differentiation, optimized channel dimensions for appropriate degree of diffusion during multi-organoid co-culture are required. It seems that in the wider diffusion channel device (ii) and the widest diffusion channel device (iii) used as control groups for the microfluidic device [standard diffusion channel (i)] developed in the present disclosure, excessive diffusion of the culture media among the culture chambers of the four different types of organoids occurred and affected proliferation and differentiation of the peripheral organoids, which made it difficult to achieve appropriate co-culture.

### Test Example 5: Verification of Possibility of Multi-Organoid Culture in Multi-Organ Microfluidic Device

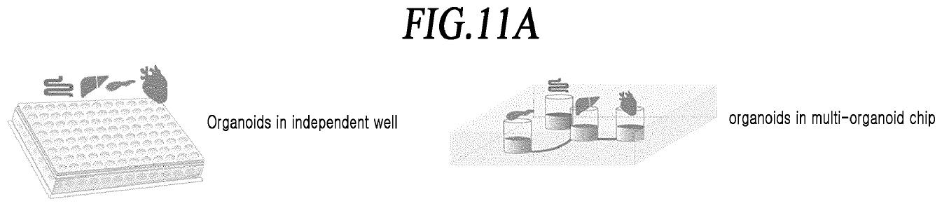

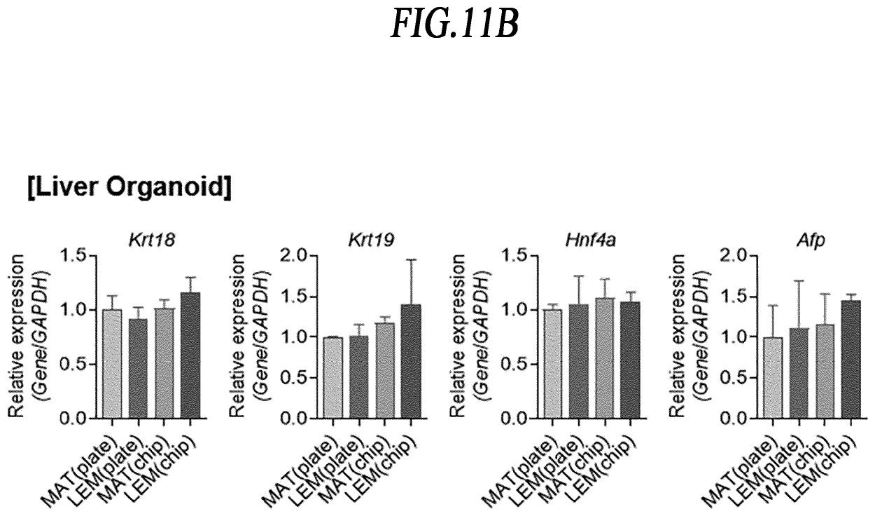

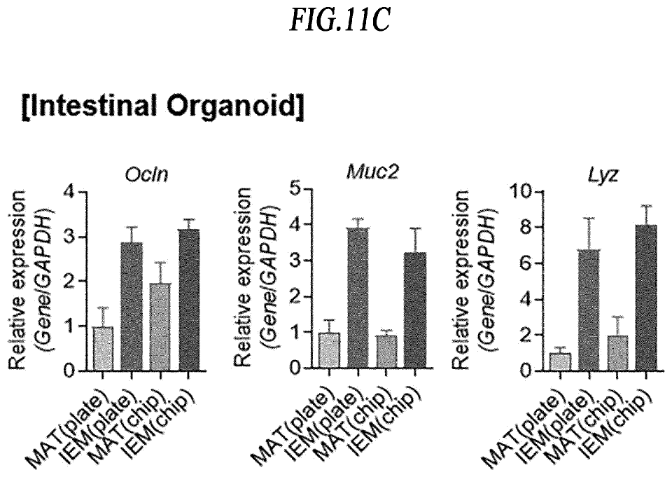

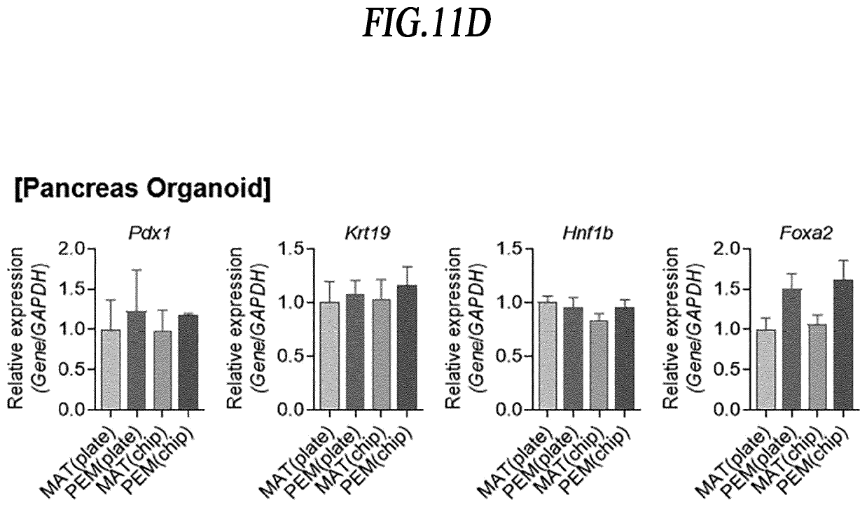

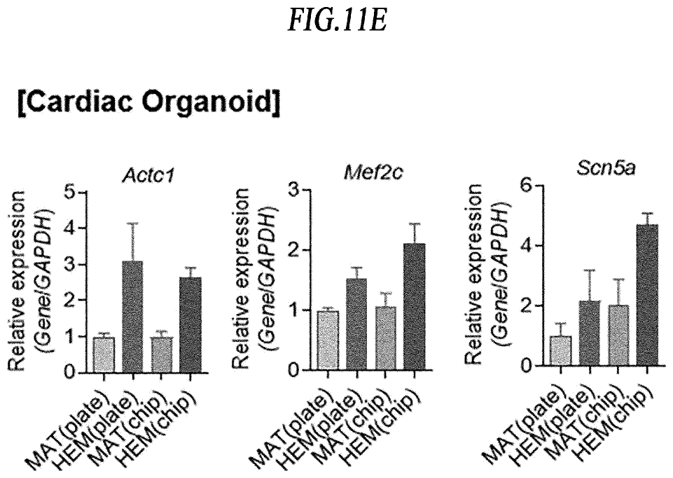

The expression of each organ-specific differentiation marker was compared by comparing liver, intestinal, pancreas and cardiac organoids each cultured in a plate. In addition to the comparison between well-plate and multi-organ microfluidic chip conditions, there was a comparison between culture in Matrigel (MAT), a commercially available culture scaffold mainly used for conventional organoid culture, and culture in an organ-specific decellularized matrix. As for MAT (plate) and MAT (chip) multi-organoids, each organoid was initially cultured in Matrigel and seeded during subculture, and as for LEM (plate) and LEM (chip) multi-organoids, organoids were initially cultured in each organ-specific decellularized matrix and seeded during subculture. The gene expression was compared by quantitative PCR analysis on day 4 of culture after the organoids were seeded into each culture platform (FIG. 11A).

As a result of comparing differentiation markers of liver organoids by quantitative PCR analysis, it was confirmed that no significant difference in the expression of Krt18 and Hnf4a was observed in the organoids of the four groups, but Krt19 and Afp had the highest expression levels in the group in which liver organoids were cultured in the decellularized liver tissue-derived matrix (6 mg/ml LEM) in the multi-organ microfluidic device (FIG. 11B).

As a result of comparing differentiation markers of intestinal organoids by quantitative PCR analysis, it was confirmed that the expression levels of a tight junction marker, Ocln, and intestinal differentiation markers, Muc2 and Lyz, were similar in the organoids cultured under the MAT (plate) and MAT (chip) conditions or slightly higher in the organoid cultured under the MAT (chip) conditions. It was confirmed that the expression levels of differentiation markers were significantly higher overall in the intestinal organoids cultured under the decellularized intestinal tissue-derived matrix (2 mg/ml IEM)-based IEM (plate) and IEM (chip) conditions than in the intestinal organoid cultured in MAT, a commercially available culture scaffold (FIG. 11C).

As a result of comparing differentiation markers of pancreas organoids by quantitative PCR analysis, it was confirmed that no significant difference in the expression of Krt19 and Hnf1b was observed in the organoids of the four groups, but Pdx1 and Foxa2 showed higher expression levels in the pancreas organoids cultured in the well-plate and the decellularized pancreas tissue-derived matrix (4 mg/ml PEM) in the multi-organ microfluidic device than in the pancreas organoid cultured in Matrigel under each condition (FIG. 11D).

As a result of comparing differentiation markers of cardiac organoids by quantitative PCR analysis, it was confirmed that the expression levels of cardiac differentiation markers, Actc1, Mef2c and Scn5a, were significantly higher in the cardiac organoid cultured in the decellularized heart tissue-derived matrix (5 mg/ml HEM) than in the cardiac organoid cultured under the MAT conditions, and the expression levels of the differentiation markers was similar in the organoids cultured under the HEM (plate) and HEM (chip) conditions or higher in the organoid cultured under the HEM (chip) conditions (FIG. 11E).

Accordingly, it was confirmed that the conventional culture scaffold can be replaced by a decellularized matrix specific to each organ, and organoids can be cultured well without a decrease in differentiation and functionality of each organoid when the organoids are co-cultured in a multi-organ microfluidic chip in the same way as when each organoid is independently cultured in a well-plate.

### Test Example 6: Comparison of Non-Alcoholic Steatohepatitis Multi-Organ Model

A multi-organ NASH model was constructed by applying not only conventional Matrigel, a commercially available culture scaffold, but also a decellularized tissue-derived hydrogel scaffold to culture of each organoid in a multi-organ device. Liver and pancreas organoids were obtained by extracting adult stem cells from a mouse tissue, encapsulated in a decellularized liver tissue-derived matrix (6 mg/ml) and a decellularized pancreas tissue-derived matrix (4 mg/ml), respectively, at a cell density of 70,000 cells/30 μL gel, and cultured. An intestinal organoid was obtained by extracting intestinal crypts from a mouse intestinal tissue and encapsulated in a decellularized intestinal tissue-derived matrix (2 mg/ml) at a cell density of 800 crypts/30 μL gel, and cultured. A cardiac organoid composed of cardiomyocytes was prepared by three-dimensionally culturing mouse embryonic fibroblasts in microwells, followed by direct reprogramming chemically induced by the culture medium components. The cardiac organoid was cultured on a hydrogel (20 organoids/70 μL gel bed) prepared by crosslinking of a decellularized cardiac tissue-derived matrix (5 mg/ml) in the form of a 70 μL gel bed at the bottom of the device. The decellularized matrix for culturing each organoid was applied at the optimal concentration for differentiation of each organoid as determined through previous research.

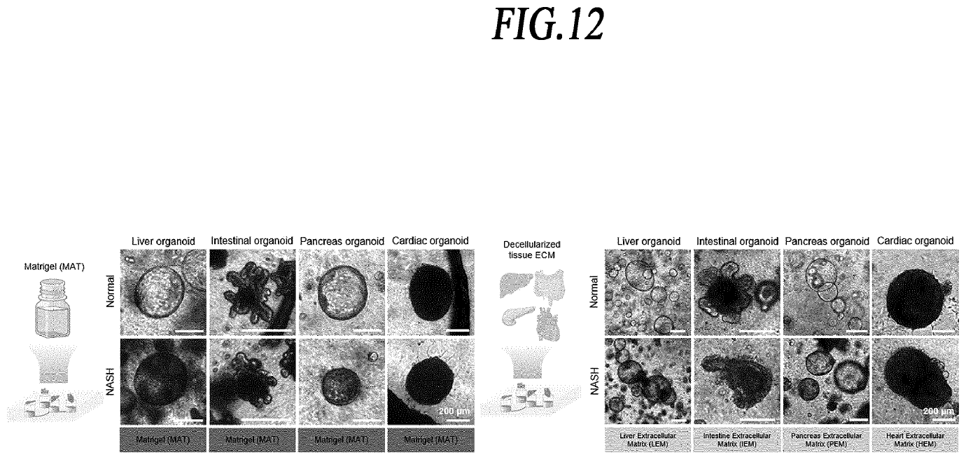

In the multi-organ microfluidic device, steatohepatitis was induced by treating the liver organoid with oleic acid (500 μM), which is a fatty acid, for 3 days, and the changes of and effects on the peripheral organoids were compared on day 3. When the organoids were cultured by using the decellularized tissue-derived hydrogel in the multi-organ NASH model, inflammation and fat accumulation occurred in the liver organoid in the same way as when the organoids were cultured by using Matrigel (MAT). The intestinal organoid as a peripheral organ had damage to the gut barrier of the intestinal tissue, and the pancreas organoid was changed in shape and induced with internal inflammation. The cardiac organoid was changed in shape as the conditions got worse or structurally deformed as cardiomyocytes inside the organoid extended into the matrix (FIG. 12).

### Test Example 7: Analysis of Effect on Peripheral Organ in Multi-Organ Non-Alcoholic Steatohepatitis Model—Culture in Matrigel

While each organoid was cultured under the Matrigel conditions in a multi-organ chip, steatohepatitis was induced by treating the liver organoid with oleic acid (500 μM), which is a type of free fatty acid, for 3 days, and the effects on the peripheral organoids were compared on day 3 after treatment with the fatty acid by comparing the expression of each marker by immunostaining. A multi-organ device in which a normal liver organoid (Normal) not treated with the fatty acid was used as a control group.

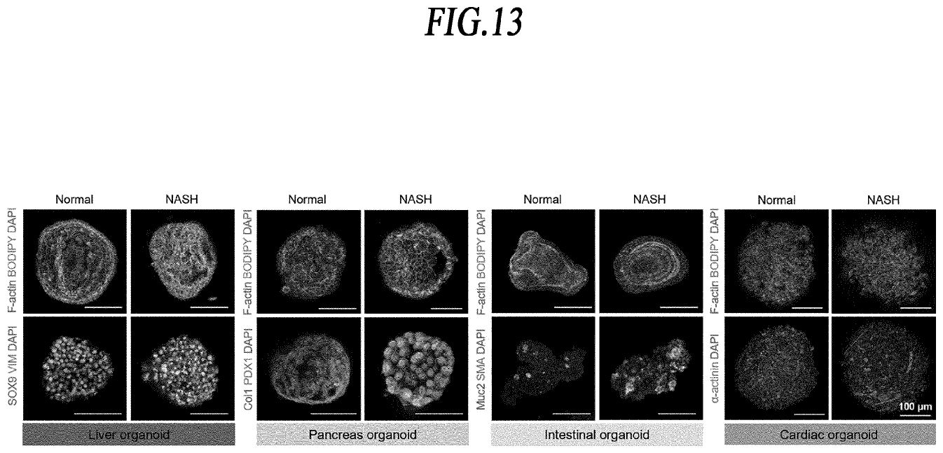

As shown in FIG. 13, as a result of checking the area where a large amount of the fatty acid was accumulated by BODIPY staining after treatment of the liver organoid with the fatty acid for 3 days, it was confirmed that fat was accumulated in a larger amount in the organoid group induced with steatohepatitis than in the normal liver organoids, but free fatty acid did not flow through the device channel to the peripheral organoids (pancreas, intestine, and heart). The peripheral organoids were affected only by paracrine factors secreted from the disease-induced organoids. As for pancreas organoids, it was confirmed that the expression level of PDX1, a pancreatic differentiation marker, was lower in the multi-organ NASH model than in the normal group and the expression level of COL1, a fibrosis marker, was greatly increased in the pancreas organoids of the multi-organ NASH model. As for intestinal organoids, it was confirmed that the MUC2, a differentiation marker, was well expressed, but SMA, a fibrosis marker, was expressed only in the intestinal organoids of the multi-organ NASH model. As for cardiac organoids, it was confirmed that the expression levels of α-actinin, a differentiation marker, and F-actin, a cytoskeletal marker, were significantly lower in the multi-organ NASH model than in the normal group.

These results demonstrate that when a fatty liver organoid model is induced in a multi-organ chip, not only a liver organoid but also the peripheral organs are affected, and, thus, the fatty liver organoid model serves as a disease model platform that reflects fluidal interactions between tissues. Therefore, it is expected that in vivo drug response and efficacy/toxicity can be evaluated more accurately than when a single fatty liver organoid model is applied.

### Test Example 8: Analysis of Effect on Peripheral Organ in Multi-Organ Non-Alcoholic Steatohepatitis Organoid Model—Culture in Decellularized Tissue-Derived Scaffold

In a decellularized tissue-derived scaffold-based multi-organ chip, steatohepatitis was induced by treating the liver organoid with oleic acid (500 μM), which is a fatty acid, for 3 days, and the effects on the peripheral organoids were compared on day 3 after treatment with the fatty acid by comparing the expression of each marker by immunostaining. A multi-organ device in which a normal liver organoid (Normal) not treated with the fatty acid was used as a control group.

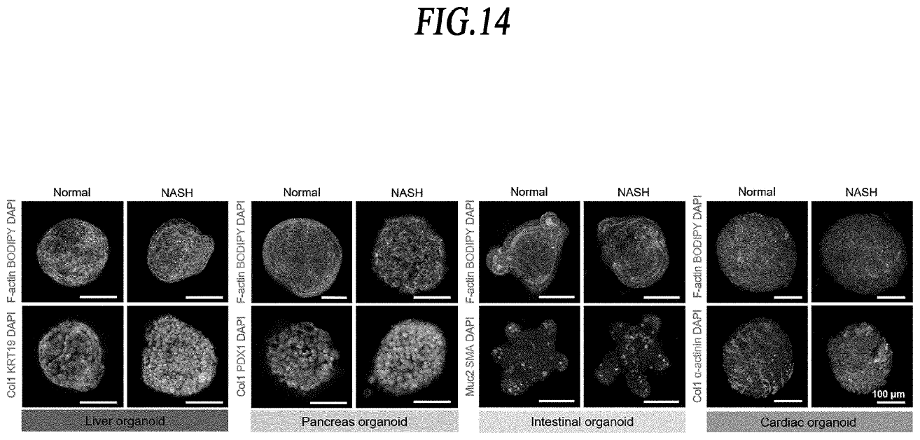

As shown in FIG. 14, as a result of checking the area where a large amount of the fatty acid was accumulated by BODIPY staining after treatment of the liver organoid with the fatty acid for 3 days, it was confirmed that fat was accumulated in a larger amount in the organoid group induced with steatohepatitis than in the normal liver organoids, but free fatty acid did not flow through the device channel to the peripheral organoids (pancreas, intestine, and heart). The peripheral organoids were affected only by paracrine factors secreted from the disease-induced organoids. As for pancreas organoids, it was confirmed that the expression level of PDX1, a pancreatic differentiation marker, was lower in the multi-organ NASH model than in the normal group and the expression level of COL1, a fibrosis marker, was greatly increased in the pancreas organoids of the multi-organ NASH model. As for intestinal organoids, it was confirmed that the MUC2, a differentiation marker, was well expressed, but SMA, a fibrosis marker, was expressed only in the intestinal organoids of the multi-organ NASH model. As for cardiac organoids, it was confirmed that the expression levels of α-actinin, a differentiation marker, and F-actin, a cytoskeletal marker, were significantly lower in the multi-organ NASH model than in the normal group, and COL1, a fibrosis marker, was expressed only in the multi-organ NASH model.

These results demonstrate that when a fatty liver organoid model is induced in a decellularized tissue-derived scaffold-based multi-organ chip, not only a liver organoid but also the peripheral organs are affected, and, thus, the fatty liver organoid model serves as a disease model platform that reflects fluidal interactions between tissues. Therefore, it is expected that in vivo drug response and efficacy/toxicity can be evaluated more accurately than when a single fatty liver organoid model is applied.

### Test Example 9: Analysis of Effect on Peripheral Organ in Multi-Organ Non-Alcoholic Steatohepatitis Organoid Model—Culture in Decellularized Tissue-Derived Scaffold

In a decellularized tissue-derived scaffold-based multi-organ chip, steatohepatitis was induced by treating the liver organoid with oleic acid (500 μM), which is a fatty acid, for 3 days, and the effects on the peripheral organoids were compared on day 3 after inducement of steatohepatitis by comparing the expression of each marker by quantitative PCR analysis. A multi-organ device in which a normal liver organoid (Normal) not treated with the fatty acid was used as a control group.

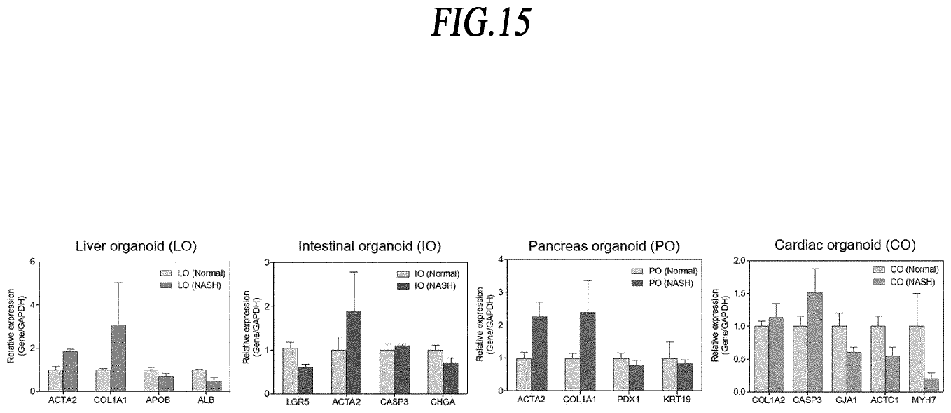

As shown in FIG. 15, it was confirmed that in the fatty liver organoid group treated with the fatty acid in the multi-organ NASH model, the expression levels of fatty liver/fibrosis markers (ACTA2 and COL1A1) increased, and the expression levels of a low-density cholesterol synthesis activity-related marker (APOB) and a mature hepatocyte marker (ALB) decreased. As for intestinal organoids, it was confirmed that the expression levels of a stemness-related marker (LGR5) and an enteroendocrine cell differentiation marker (CHGA) decreased and the expression level of ACTA2, a fibrosis-related marker, and CASP3, an apoptosis-related marker, increased in the multi-organ NASH model. Also, as for pancreas organoids, it was confirmed that the expression levels of ACTA2 and COL1A2, fibrosis markers, increased and the expression levels of PDX1 and KRT19, pancreatic differentiation markers, were similar or decreased in the multi-organ NASH model. As for cardiac organoids of the multi-organ NASH model, it was confirmed that the expression levels of COL1A2, a fibrosis marker, and CASP3, an apoptosis marker, increased and the expression levels of GJA1, ACTC1 and MYH7, cardiac differentiation markers, greatly decreased.

These results demonstrate that when a fatty liver organoid model is induced in a decellularized tissue-derived scaffold-based multi-organ chip, not only a liver organoid but also the peripheral organs are affected, and, thus, the fatty liver organoid model serves as a disease model platform that reflects fluidal interactions between tissues. Therefore, it is expected that in vivo drug response and efficacy/toxicity can be evaluated more accurately than when a single fatty liver organoid model is applied.

### Test Example 10: Analysis of NASH Therapeutic Effect of Candidate Drug in Multi-Organ Non-Alcoholic Steatohepatitis Organoid Model (Liver Organoid)

In a decellularized tissue-derived scaffold-based multi-organ chip, steatohepatitis was induced by treating the liver organoid with a fatty acid, and the effects of four representative drugs, which have been recently eliminated after entering clinical trials as candidate drugs for non-alcoholic steatohepatitis, on the liver organoid were evaluated. Obeticholic acid (OCA), a semi-synthetic bile acid analog, is an agonist for FXR (farnesoid X receptor) to regulate bile acid metabolism, inflammation, fibrosis, and sugar/lipid metabolism. Ezetimibe (Eze), a therapeutic drug for hyperlipidemia, is a drug that selectively inhibits cholesterol absorption in the small intestine. Elafibranor (Ela), a dual agonist for PPARα/δ, is a drug that inhibits fatty acid biosynthesis and glucose biosynthesis in the liver and has been used as a drug for cardiometabolic diseases. Liraglutide (Lira), an agonist for glucagon-like peptide-1 (GLP-1), is a drug that has been used to treat diabetes. Each organoid cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

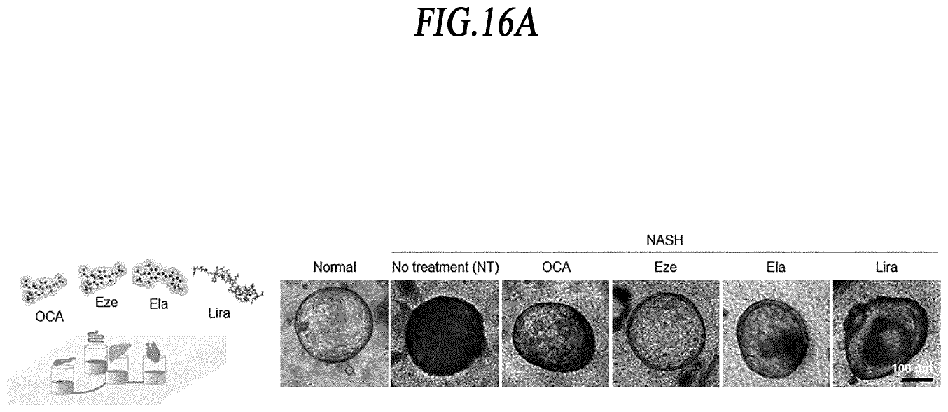

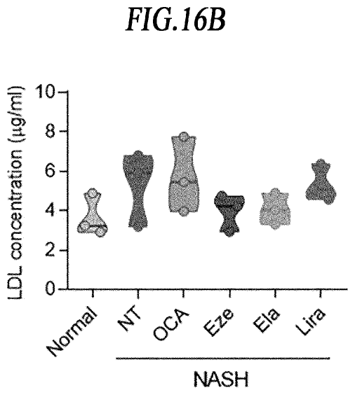

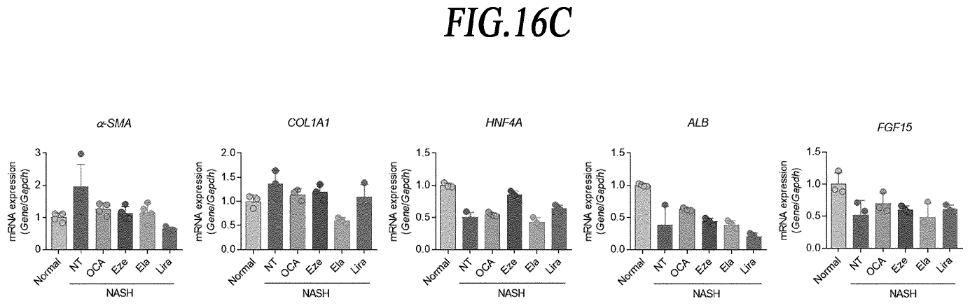

As a result of optical microscopy on each group, it was confirmed that the liver organoids in the Normal group were cultured normally without internal inflammation or fatty acid accumulation. Also, it was confirmed that in the NASH-induced group, the inside of the organoids turned dark due to inflammation and fatty acid accumulation, and in the groups treated with the four candidate drugs whose efficacy in improving non-alcoholic steatohepatitis was demonstrated to some extent, inflammation and fatty acid accumulation were partially improved (FIG. 16A).

Further, the liver organoids of each group were disrupted, followed by cholesterol analysis to check whether the four candidate drugs are effective in improving low-density cholesterol (LDL) levels in the liver organoids. As a result, it was confirmed that the LDL levels in an OCA-treated group were similar to or higher than those in a No Treatment (NT) group, which confirmed that an increase of in vivo LDL level, which is known as a side effect of OCA, appeared in the multi-organ NASH model. Meanwhile, Eze is known as being effective in reducing LDL by acting as a cholesterol absorption inhibitor in the liver and intestine, and the LDL levels in an Eze-treated group were significantly lower than those in the No Treatment (NT) group. Ela and Lira also showed an improvement in LDL levels compared to the No Treatment (NT) group, and it was confirmed that Ela showed a greater improvement than Lira (FIG. 16B).

Furthermore, as a result of comparing the gene expression of each group by quantitative PCR analysis, it was confirmed that α-SMA and COL1A1, fibrosis and drug toxicity-related markers, were increased in the NASH group compared to the normal group, and improved by treatment with OCA, Eze, Ela, and Lira (FIG. 16C). In particular, it was confirmed that Lira is effective in reducing an increase of α-SMA caused by NASH, and Ela is remarkably effective in reducing the increased expression of COL1A1. Also, it was confirmed that the expression levels of HNF4A and ALB, hepatic differentiation markers, were remarkably decreased in the NASH-induced liver organoid group, and the expression levels of the hepatic differentiation marker were recovered to some extent in the OCA- and Eze-treated groups. However, it was confirmed that Ela and Lira have no significant effect on hepatic differentiation and functional recovery. FGF15 is highly expressed in intestinal tissue in liver-intestine interactions and is known as an endocrine factor that regulates bile acid synthesis in the liver. The expression of FGF15 is regulated by FXR (Farnesoid X receptor), and OCA acts as an FXR agonist and is known as recovering the decreased expression of FGF15 in the intestine and liver due to NASH. Actually, in the developed multi-organ NASH model, a remarkable decrease in the gene expression of FGF15 in the NASH-induced liver organoid compared to the normal group and a certain improvement caused by treatment with the candidate drugs were observed. In particular, the gene expression of FGF15 was recovered the most in the organoids of the OCA-treated group.

These results demonstrate that the multi-organ non-alcoholic steatohepatitis organoid model prepared in the present disclosure can mimic the effect of drug treatment in an actual NASH patient in vitro, and can serve as a disease model platform that reflects fluidal interactions between organs.

Further, in a decellularized tissue-derived scaffold-based multi-organ chip, steatohepatitis was induced by treating the liver organoid with a fatty acid, and the effects of four representative drugs, which have been recently eliminated after entering clinical trials as candidate drugs for non-alcoholic steatohepatitis, on the liver organoid were evaluated by immunostaining for key markers. Each organoid cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

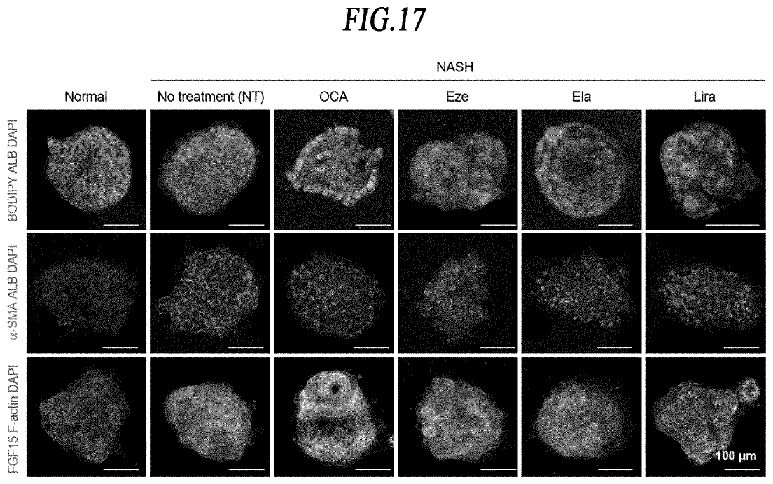

As a result of checking the expression of key markers in the liver organoids by immunofluorescence staining analysis, it was confirmed as shown in FIG. 17 that the expression level of ALB, a hepatic differentiation marker, was high and fatty acid accumulation did not occur in the normal liver organoids, whereas many parts expressing BODIPY (parts where a large amount of the fatty acid was accumulated) due to inducement of fatty liver were observed in the liver organoids of the NASH group. However, it was confirmed that in all of the groups treated with the candidate drugs for NASH, fatty acid accumulation was improved to some extent. Also, it was confirmed that the expression level of α-SMA, a fibrosis marker, was the highest in the NASH group, and decreased in the groups treated with the four candidate drugs known as being effective in improving steatohepatitis, which confirmed that fibrosis caused by steatohepatitis was improved by treatment with the drugs. In particular, it was confirmed that fibrosis was improved the most in the Ela- and Lira-treated groups. FGF15 is highly expressed in intestinal tissue in liver-intestine interactions and is known as an endocrine factor that regulates bile acid synthesis in the liver. The expression of FGF15 is regulated by FXR (Farnesoid X receptor), and OCA acts as an FXR agonist and is known as recovering the decreased expression of FGF15 in the intestine and liver due to NASH. Actually, as a result of immunostaining in the developed multi-organ NASH model, it was confirmed that the expression level of FGF15 was high in the normal liver organoids, but decreased in the NASH-induced organoids and recovered only in the OCA-treated group.

These results demonstrate that the multi-organ non-alcoholic steatohepatitis organoid model prepared in the present disclosure can mimic the effect of drug treatment on an actual NASH patient in vitro, and can serve as a disease model platform that reflects fluidal interactions between organs. Therefore, it is expected that in vivo drug response and efficacy/toxicity can be evaluated more accurately than when a single fatty liver organoid model is applied.

### Test Example 11: Analysis of Effect of Candidate Drug on Peripheral Organ in Multi-Organ Non-Alcoholic Steatohepatitis Organoid Model (Intestinal Organoid)

In a decellularized tissue-derived scaffold-based multi-organ chip, steatohepatitis was induced by treating the liver organoid with a fatty acid, and the effects of four representative drugs, which have been recently eliminated after entering clinical trials as candidate drugs for non-alcoholic steatohepatitis, on the intestinal organoid were evaluated. Each organoid cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

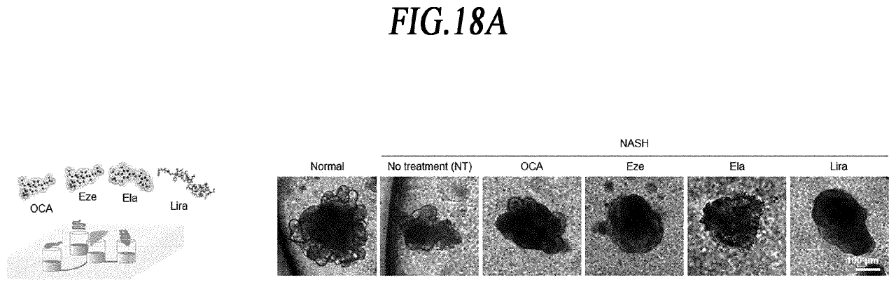

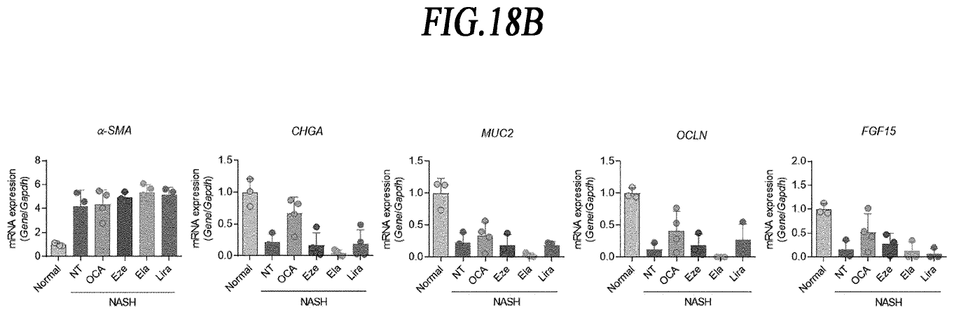

As a result of optical microscopy on each group, it was confirmed that the gut barrier of the intestinal organoid was well maintained in the Normal group and gut organoid-specific budding was observed (FIG. 18A). As for the NASH group, it was confirmed in the No Treatment (NT) group and the Ela-treated group, the gut barrier of the intestinal organoid collapsed or burst and no budding occurred, whereas in the Eze- and Lira-treated groups, the gut barrier was maintained to some extent but organoid budding hardly occurred. Meanwhile, it was confirmed that in the OCA-treated group, the gut barrier of the intestinal organoid was maintained to some extent and some budding occurred.

As a result of comparing the gene expression of the intestinal organoids of the groups treated with the respective drugs by quantitative PCR analysis, it was confirmed as shown in FIG. 18B that the expression level of α-SMA, a fibrosis and drug toxicity-related marker, was greatly increased by inducement of NASH compared to the normal group, and the increased expression level of α-SMA was not decreased even in the respective groups treated with the four candidate drugs for NASH. Accordingly, it was confirmed that the drugs were effective in improving fibrosis of steatohepatitis, but not much effective in reducing toxicity to the intestinal organoids and improving fibrosis. Also, it was confirmed that the expression levels of CHGA and

MUC2, intestinal differentiation markers, were decreased remarkably in the intestinal organoids of the NASH-induced No Treatment (NT) group, and recovered to some extent in the OCA-treated group. Meanwhile, it was confirmed that the expression levels of the differentiation markers were not much recovered in the Eze-, Ela- and Lira-treated groups, and in particular, the expression levels of the differentiation markers were lower in the Ela-treated group than in No Treatment (NT) group. When NASH is induced, the gut barrier of the intestine collapses and the permeability is greatly affected. Therefore, the gene expression for OCLN, a tight junction marker of the gut barrier, was compared. As a result, it was confirmed that the expression level of OCLN was decreased greatly in the No Treatment (NT) group, and recovered to some extent in the OCA-, Eze- and Lira-treated groups, but not improved in the Ela-treated group. FGF15 is highly expressed in intestinal tissue in liver-intestine interactions and is known as an endocrine factor that regulates bile acid synthesis in the liver. The expression of FGF15 is regulated by FXR (Farnesoid X receptor), and OCA acts as an FXR agonist and is known as recovering the decreased expression of FGF15 in the intestine and liver due to NASH. Actually, when NASH was induced, the gene expression of FGF15 in the intestinal organoid decreased in the multi-organ-based NASH organoid model, but was recovered to some extent in the Eze-treated group and recovered to a significant extent in the OCA-treated group.

These results demonstrate that the multi-organ non-alcoholic steatohepatitis organoid model prepared in the present disclosure can mimic the effect of drug treatment on other organs in an actual NASH patient in vitro, and can serve as a disease model platform that reflects fluidal interactions between organs.

Further, in a decellularized tissue-derived scaffold-based multi-organ chip, steatohepatitis was induced by treating the liver organoid with a fatty acid, and the effects of four representative drugs, which have been recently eliminated after entering clinical trials as candidate drugs for non-alcoholic steatohepatitis, on the liver organoid were evaluated by immunostaining for key markers. Specifically, each organoid cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

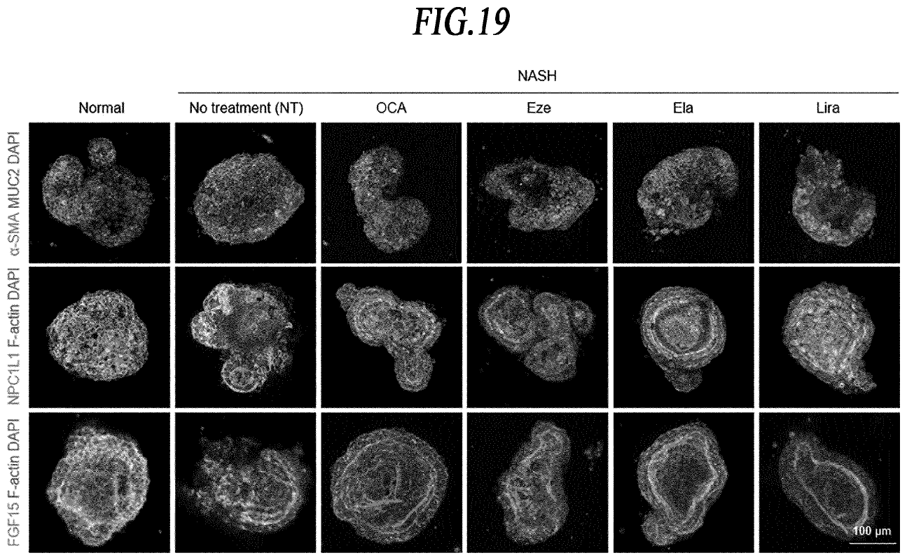

As a result of checking the expression of key markers in the intestinal organoids by immunofluorescence staining analysis, it was confirmed as shown in FIG. 19 that the expression level of MUC2, an intestinal differentiation marker, was the highest in the Normal group and decreased in the NASH-induced group. Also, it was confirmed that the expression level of the differentiation marker was improved to some extent only in the OCA-treated group among the groups treated with the four types of drugs, and there was no significant difference in the expression of MUC2 among the groups treated with the other groups. Further, it was confirmed that the expression level of α-SMA, a toxicity and fibrosis-related marker, increased overall in the intestinal organoids in the NASH-induced group compared to the normal group. However, the expression level of α-SMA in the intestinal organoids was not decreased by the four candidate drugs that were effective in improving steatohepatitis, which confirmed that there is a limit to the efficacy of these drugs in improving toxicity to the intestinal organoids and fibrosis. NPC1L1 is a protein expressed in gastrointestinal epithelial cells and hepatocytes and binds to a critical mediator of cholesterol absorption. Eze has been selected as a candidate drug for NASH by inhibiting NPC1L1 and reducing cholesterol absorption in the liver and intestine. As a result of immunostaining for NPC1L1, it was confirmed that the expression of NPC1L1 was hardly observed in the intestinal organoids of the Normal group, but increased due to an increase in cholesterol absorption in the intestine when NASH was induced. Also, it was confirmed that NPC1L1 showed the most significant decrease in expression in the intestinal organoids of the Eze-treated group. As a result of comparing the expression of FGF15 in the intestinal organoids in the same way as for the liver organoids, it was confirmed that the expression level of FGF15 was decreased in the NASH-induced intestinal organoids and recovered to some extent in the intestinal organoids of the OCA-treated group. This result shows a tendency that almost coincides with the result of analysis of the liver organoids.

These results demonstrate that the multi-organ non-alcoholic steatohepatitis organoid model prepared in the present disclosure can mimic the effect of drug treatment on other organs in an actual NASH patient in vitro, and can serve as a disease model platform that reflects fluidal interactions between organs.

### Test Example 12: Analysis of Effect of Candidate Drug on Peripheral Organ in Multi-Organ Non-Alcoholic Steatohepatitis Organoid Model (Pancreas Organoid)

In a decellularized tissue-derived scaffold-based multi-organ chip, steatohepatitis was induced by treating the liver organoid with a fatty acid, and the effects of four representative drugs, which have been recently eliminated after entering clinical trials as candidate drugs for non-alcoholic steatohepatitis, on the pancreas organoid were evaluated. Each organoid cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

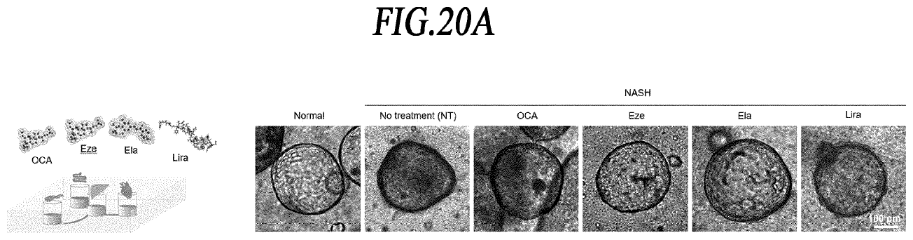

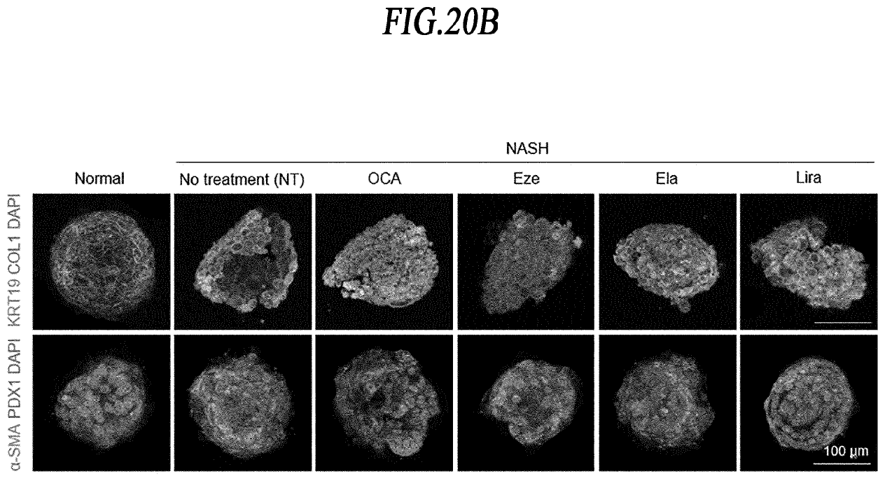

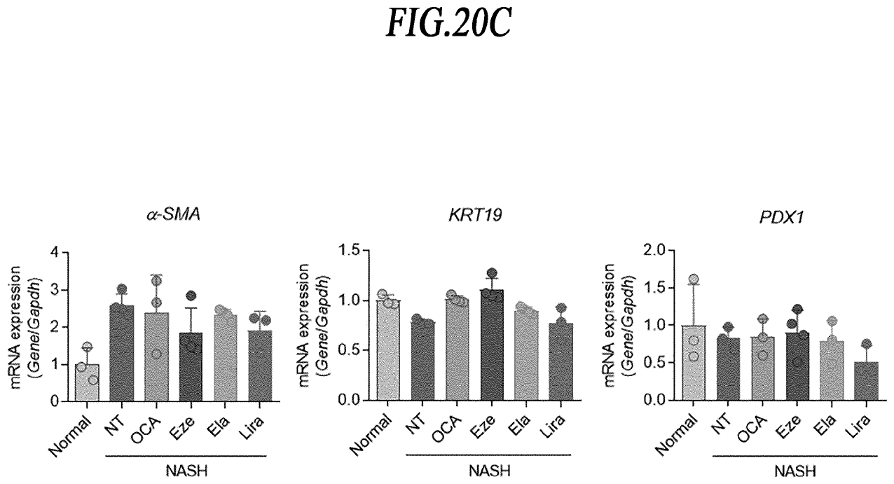

As a result of optical microscopy on each group, it was confirmed as shown in FIG. 20A that a cystic shape specific to the pancreas organoid was well observed in the Normal group. However, it was confirmed that in the NASH-induced No Treatment (NT) group and the OCA-, Ela- and Lira-treated groups, the epithelial lumen of the pancreas organoids increased in thickness, showing the pattern of early fibrosis. It was confirmed that the epithelial lumen was maintained at a normal level in the Eze-treated group compared to the groups treated with the other drugs.

As a result of immunostaining of each group, it was confirmed that the expression levels of KRT19 and PDX1, pancreatic differentiation markers, were higher in the Normal group than in the other groups, and α-SMA and COL1, fibrosis and drug toxicity-related markers, were hardly expressed. However, it was confirmed that in the NASH-induced No Treatment (NT) group, the expression levels of KRT19 and PDX1 decreased and the expression levels of α-SMA and COL1 greatly increased. The expression levels of α-SMA and COL1 were significantly decreased in the Eze-treated group among the groups treated with the candidate drugs, and the groups treated with the other groups did not show remarkable improvement in fibrosis and recovery of expression of differentiation/functional markers (FIG. 20B).

Furthermore, as a result of comparing the gene expression of the pancreas organoids of the groups treated with the respective drugs by quantitative PCR analysis, it was confirmed as shown in FIG. 20C that the expression level of α-SMA, a fibrosis and drug toxicity-related marker, was greatly increased in the NASH group compared to the normal group, and pancreatitis and fibrosis were reduced in the Eze- and Lira-treated groups. However, in the OCA- and Ela-treated groups, the expression level of α-SMA was not decreased, which confirmed that there was some drug toxicity in the pancreas organoids. Also, it was confirmed that the expression levels of KRT19 and PDX1, pancreas-specific differentiation markers, decreased in the No Treatment (NT) group, resulting in a decrease in pancreatic differentiation and functionality, but in the OCA-, Eze- and Lira-treated groups, the expression level of KRT19, a pancreatic exocrine cell marker, was recovered to some extent and the expression level of PDX1, an endocrine cell marker, was not recovered. Further, it was confirmed that Lira has limitations in recovering both exocrine and endocrine function-related markers.

These results demonstrate that the multi-organ non-alcoholic steatohepatitis organoid model prepared in the present disclosure can mimic the effect of drug treatment on other organs in an actual NASH patient in vitro, and can serve as a disease model platform that reflects fluidal interactions between organs.

### Test Example 13: Analysis of Effect of Candidate Drug on Peripheral Organ in Multi-Organ Non-Alcoholic Steatohepatitis Organoid Model (Cardiac Organoid)

In a decellularized tissue-derived scaffold-based multi-organ chip, steatohepatitis was induced by treating the liver organoid with a fatty acid, and the effects of four representative drugs, which have been recently eliminated after entering clinical trials as candidate drugs for non-alcoholic steatohepatitis, on the cardiac organoid were evaluated. Each organoid cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber were cultured by treating with oleic acid (500 μM) and each drug (50 μM) for 3 days in the chamber in which the liver organoid was cultured. A multi-organ device in which a normal liver organoid (Normal) not treated with the fatty acid was used as a control group.

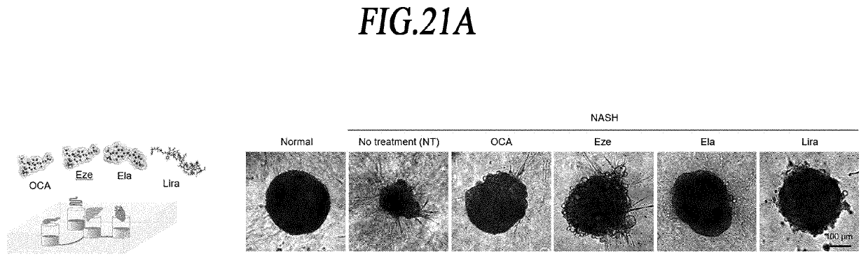

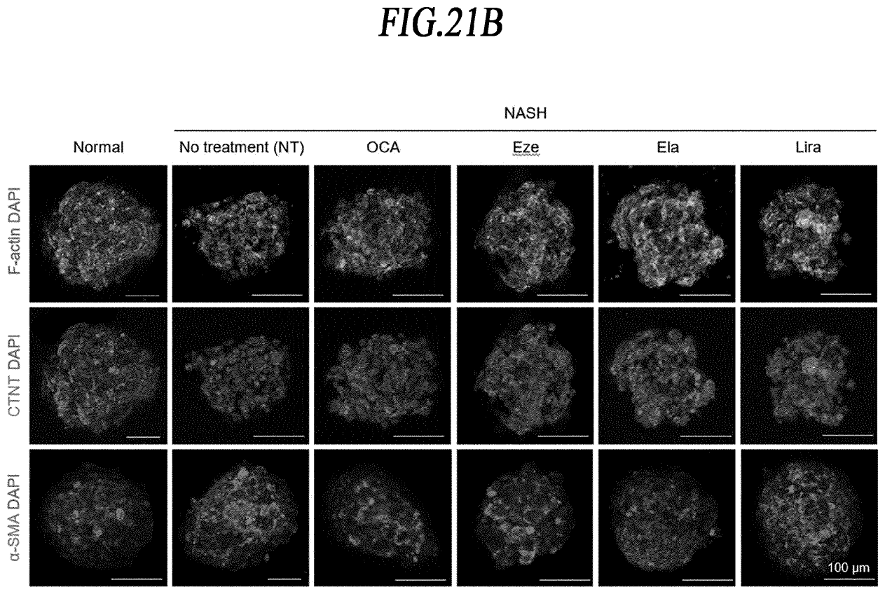

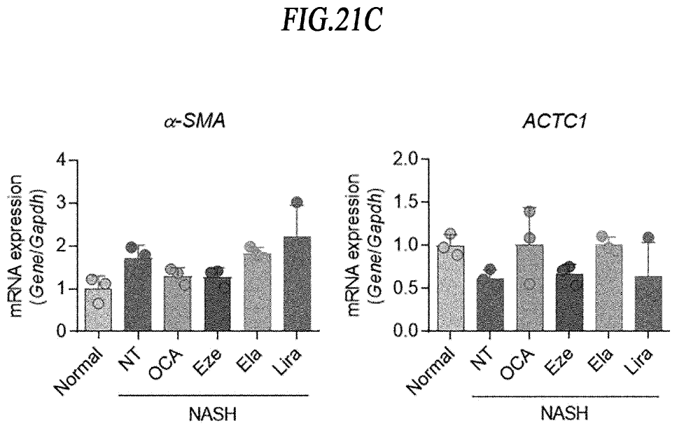

As a result of optical microscopy on each group, it was confirmed as shown in FIG. 21A that the shape of the cardiac organoids of the NASH group collapsed when the size of the cardiac organoids decreased due to inflammatory paracrine factors diffused from the steatohepatitis organoids and the cells extended out of the cardiac organoids compared to the cardiac organoids of the Normal group. Also, it was confirmed that in the OCA- and Ela-treated groups, the shape of the cardiac organoids was relatively intact, but in the Lira-treated group, some cells inside the organoids died, inducing severe cardiotoxicity.

As a result of immunostaining of each group, it was confirmed as shown in FIG. 21B that CTNT and F-actin, myocardial differentiation and actin-filament markers, were well expressed in the cardiac organoids of the Normal group and the myocardial fibers were long and thick. However, it was confirmed that in the NASH group, the expression levels of the these marker was remarkably decreased, and an abnormal pattern with broken myocardial fibers was observed. Also, it was confirmed that in the OCA- and Ela-treated groups, the expression levels of myocardial fibers and actin-filaments were recovered to some extent, but in the Lira-treated group, no recovery of expression of the cardiac differentiation marker was observed. Further, it was confirmed that the expression level of α-SMA, a fibrosis and drug toxicity-related marker, was greatly increased in the NASH-induced No Treatment group, and fibrosis was not improved even in the Ela- and Lira-treated groups. However, the expression level of α-SMA decreased in the OCA- and Eze-treated groups, which confirmed that cardiac fibrosis was improved to some extent.

As a result of comparing the gene expression of the cardiac organoids of the groups treated with the respective drugs by quantitative PCR analysis, it was confirmed as shown in FIG. 21C that the expression level of α-SMA, a fibrosis and drug toxicity-related marker, was greatly increased in the NASH group compared to the normal group. However, in the OCA- and Eze-treated groups, fibrosis was improved, and in the Lira-treated group, fibrosis tended to worsen. Also, it was confirmed that the expression level of ACTC1, a cardiac differentiation marker, was decreased in the NASH-induced No Treatment (NT) group, but recovered by treatment with OCA and Ela, which was actually used in clinical trials for cardiometabolic diseases. Further, it was confirmed that Eze and Lira have limitations in improving cardiac differentiation/functionality. These results demonstrate that the multi-organ non-alcoholic steatohepatitis

organoid model prepared in the present disclosure can mimic the effect of drug treatment on other organs in an actual NASH patient in vitro, and can serve as a disease model platform that reflects fluidal interactions between organs.

### Test Example 14: Analysis of Effect of Candidate Drug on Peripheral Organ in Human Induced Pluripotent Stem Cell (hiPSC)-Derived Multi-Organ Non-Alcoholic Steatohepatitis Organoid Model-Liver and Pancreas Organoids

Human iPSC-derived liver organoids were differentiated into hepatic endoderm cells through endoderm, and then a total of 500,000 cells including the hepatic endoderm cells, vascular endothelial cells (HUVEC) and mesenchymal stem cells (hMSC) at a ratio of 10:7:2 were mixed in 10 μL of culture medium. The mixture was coated onto 10 μL of a decellularized liver tissue-derived matrix (LEM) to induce the formation of three-dimensional organoids. Human iPSC-derived intestinal organoids were differentiated into hindgut spheroids through endoderm, encapsulated in Matrigel, and matured for about 20 to 30 days. The mature intestinal organoids were extracted from Matrigel, re-encapsulated in a decellularized intestinal tissue-derived matrix (IEM), and further cultured. Human iPSC-derived pancreas organoids were differentiated into beta cells through endoderm, gut endoderm, pancreatic endoderm, and pancreatic progenitor cells, and then a total of 500,000 cells including the beta cells, vascular endothelial cells (HUVEC) and mesenchymal stem cells (hMSC) at a ratio of 10:7:2 were mixed in 10 μL of culture medium. The mixture was coated onto 10 μL of a decellularized pancreas tissue-derived matrix (PEM) to induce the formation of three-dimensional organoids. Human iPSC-derived cardiac organoids were induced to mature into cardiomyocytes through mesenchyma and cardiac progenitor cells, and then 400,000 cells were mixed in 20 μL of a decellularized heart tissue-derived matrix (HEM) to prepare three-dimensional organoids. In a decellularized tissue-derived scaffold-based multi-organ chip, steatohepatitis was induced by treatment of the liver organoids with a fatty acid, and the effects of representative drugs, which have been recently eliminated after entering clinical trials as candidate drugs for non-alcoholic steatohepatitis, on the liver and pancreas organoids were evaluated. Each organoids cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

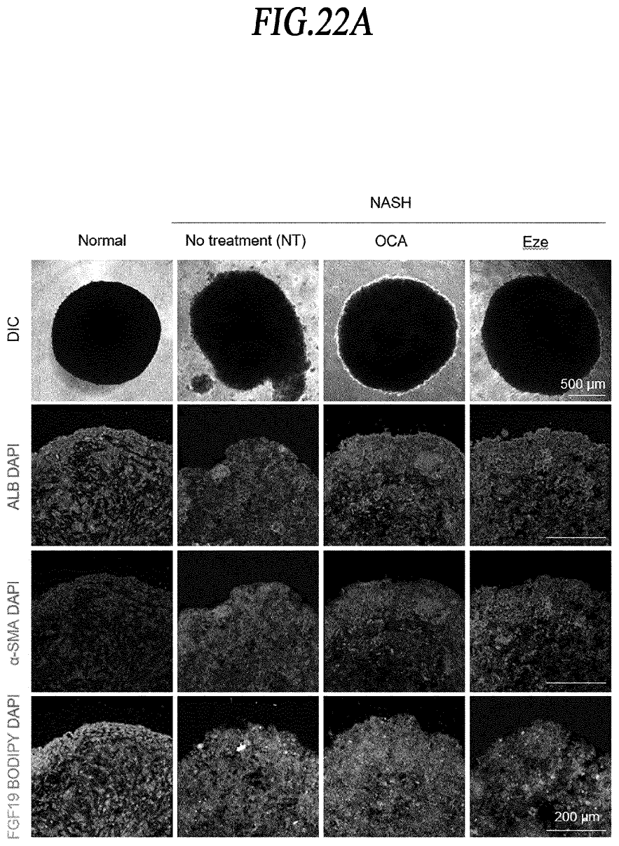

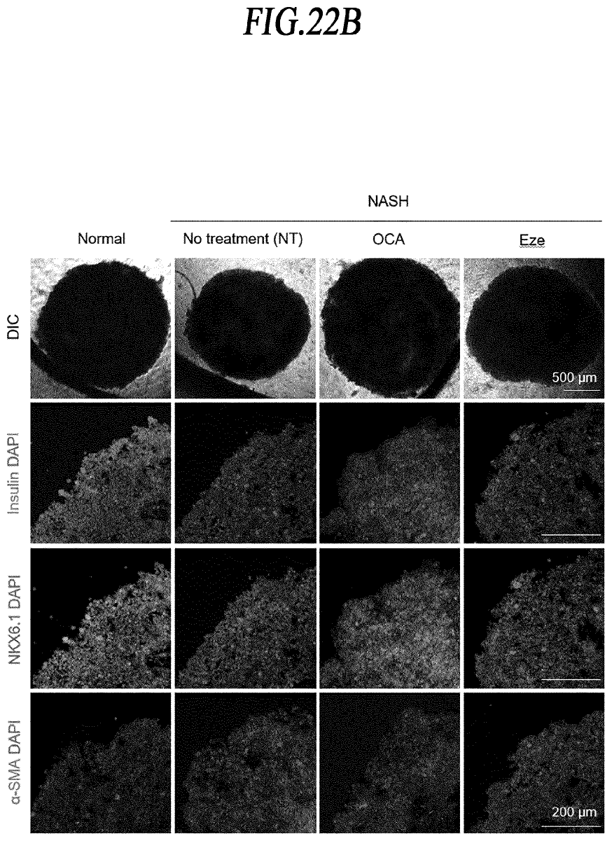

As a result of optical microscopy and immunofluorescence staining analysis of the human iPSC-derived liver organoids in each group, it was confirmed as shown in FIG. 22A that the structure of the liver organoids was best maintained in the Normal group, but in the liver organoids of the No Treatment (NT) group and the OCA- and Eze-treated groups of the NASH group, some of the cells in the outer part died. In particular, it was confirmed that the organoids of the NT (No Treatment) group did not maintain their shape well. As a result of comparison by immunofluorescence staining analysis, it was confirmed that the liver organoids of the Normal group showed a high expression level of ALB, a hepatic differentiation marker, and almost no expression of α-SMA, a fibrosis and drug toxicity-related marker. However, it was confirmed that in the liver organoids of the No Treatment group, the expression level of α-SMA greatly increased while the expression level of ALB decreased. Also, it was confirmed that in the OCA- and Eze-treated groups, the expression level of α-SMA was decreased to some extent. As a result of BODIPY staining for staining accumulated fatty acids, it was confirmed that fatty acid accumulation did not occur in the liver organoids of the Normal group, whereas a large amount of fatty acid accumulation occurred in the organoids of the No Treatment group, and in the OCA- and Eze-treated groups, fatty acid accumulation was partially decreased. FGF19 (a human-derived protein like FGF15 in the mouse organoid) is highly expressed in intestinal tissue in liver-intestine interactions and is known as an endocrine factor that regulates bile acid synthesis in the liver. The expression of FGF19 is regulated by FXR (Farnesoid X receptor), and OCA acts as an FXR agonist and is known as recovering the decreased expression of FGF19 in the intestine and liver due to NASH. It was confirmed that the expression level of FGF19 was high in the normal liver organoids and decreased significantly in the NT (No Treatment) group, but recovered only in the OCA-treated group among the OCA- and Eze-treated groups.

As a result of optical microscopy and immunofluorescence staining analysis of the human iPSC-derived pancreas organoids in each group, it was confirmed as shown in FIG. 22B that no significant difference among the groups was observed from optical microscopy images, but immunofluorescence staining showed differences among the groups. It was confirmed that the expression levels of insulin and NKX6.1, pancreatic differentiation markers, were high and the expression of α-SMA, a fibrosis and drug toxicity-related marker, was hardly observed in the pancreas organoids of the Normal group, whereas the expression levels of insulin and NKX6.1 decreased and the expression level of α-SMA greatly increased in the pancreas organoids of the No Treatment group. Meanwhile, it was confirmed that the expression levels of the differentiation markers were greatly recovered and the expression level of α-SMA was decreased in the OCA-treated group. Also, it was confirmed that the expression levels of the differentiation markers were less significantly recovered in the pancreas organoids treated with Eze than in the pancreas organoids treated with OCA-treated group, and the expression level of α-SMA was still higher in the Eze-treated group than in the OCA-treated group.

These results demonstrate that even when the multi-organ non-alcoholic steatohepatitis organoid model developed in the present disclosure is fabricated using human iPSC-derived organoids, it can mimic the effect of drug treatment on other organs in an actual NASH patient in vitro, and can serve as a disease model platform that reflects fluidal interactions between organs.

### Test Example 15: Analysis of Effect of Candidate Drug on Peripheral Organ in Human Induced Pluripotent Stem Cell (hiPSC)-Derived Multi-Organ Non-Alcoholic Steatohepatitis Organoid Model—Intestinal and Cardiac Organoids

Human iPSC-derived liver organoids were differentiated into hepatic endoderm cells through endoderm, and then a total of 500,000 cells including the hepatic endoderm cells, vascular endothelial cells (HUVEC) and mesenchymal stem cells (hMSC) at a ratio of 10:7:2 were mixed in 10 μL of culture medium. The mixture was coated onto 10 μL of a decellularized liver tissue-derived matrix (LEM) to induce the formation of three-dimensional organoids. Human iPSC-derived intestinal organoids were differentiated into hindgut spheroids through endoderm, encapsulated in Matrigel, and matured for about 20 to 30 days. The mature intestinal organoids were extracted from Matrigel, re-encapsulated in a decellularized intestinal tissue-derived matrix

(IEM), and further cultured. Human iPSC-derived pancreas organoids were differentiated into beta cells through endoderm, gut endoderm, pancreatic endoderm, and pancreatic progenitor cells, and then a total of 500,000 cells including the beta cells, vascular endothelial cells (HUVEC) and mesenchymal stem cells (hMSC) at a ratio of 10:7:2 were mixed in 10 μL of culture medium. The mixture was coated onto 10 μL of a decellularized pancreas tissue-derived matrix (PEM) to induce the formation of three-dimensional organoids. Human iPSC-derived cardiac organoids were induced to mature into cardiomyocytes through mesenchyma and cardiac progenitor cells, and then 400,000 cells were mixed in 20 μL of a decellularized heart tissue-derived matrix (HEM) to prepare three-dimensional organoids. In a decellularized tissue-derived scaffold-based multi-organ chip, steatohepatitis was induced by treatment of the liver organoids with a fatty acid, and the effects of representative drugs, which have been recently eliminated after entering clinical trials as candidate drugs for non-alcoholic steatohepatitis, on the intestinal and cardiac organoids were evaluated. Each organoids cultured in a decellularized tissue-derived matrix was seeded into a multi-organ microfluidic device and then cultured in a normal culture medium suitable for each organ for 2 days. Then, the NASH group was treated with oleic acid (500 μM) and cultured for 3 days only in the chamber in which the liver organoid was cultured, and the groups treated with the respective drugs were treated with oleic acid (500 μM) and the respective drugs (50 μM) for 3 days in the chamber in which the liver organoid was cultured. A multi-organ device in which a normal liver organoid (Normal) not treated with the fatty acid was used as a control group.

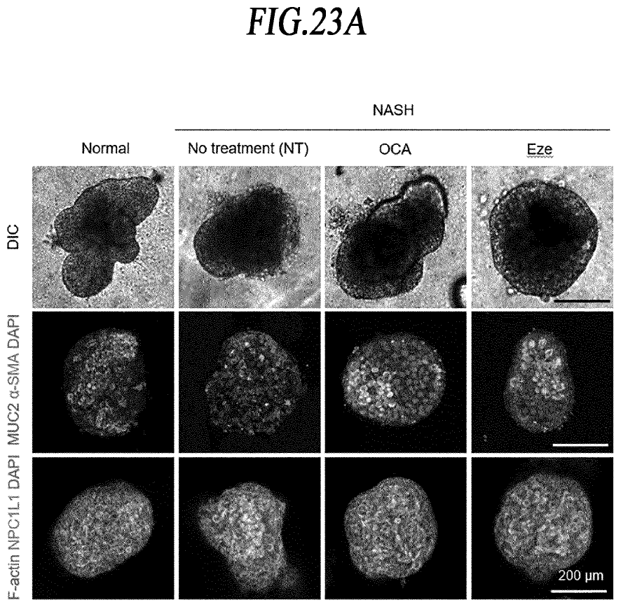

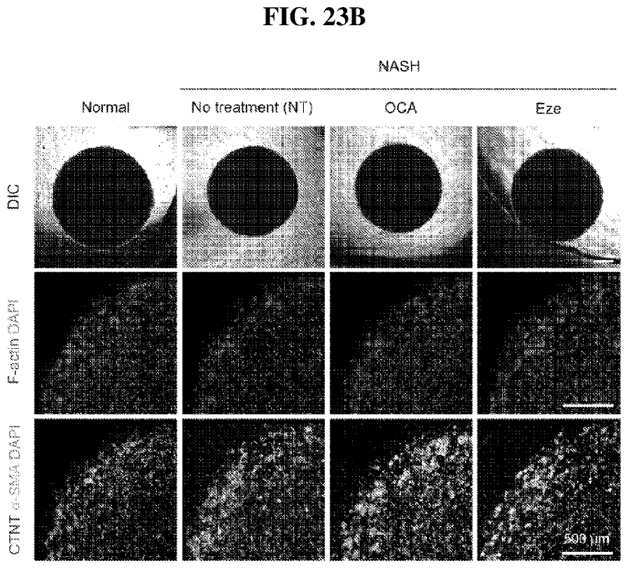

As a result of optical microscopy and immunofluorescence staining analysis of the human iPSC-derived intestinal organoids in each group, it was confirmed as shown in FIG. 23A that the gut barrier of the intestinal organoid was well maintained in the Normal group and gut organoid-specific budding was observed. Also, it was confirmed when NASH was induced, the gut barrier of the intestinal organoid collapsed or burst and no budding occurred in the No Treatment (NT) group and the gut barrier was partially damaged and budding occurred to some extent in the OCA-treated group. Also, it was confirmed that the gut barrier was maintained to some extent but organoid budding hardly occurred in the Eze-treated group. As a result of comparison by immunofluorescence staining analysis, it was confirmed that the expression level of MUC2, an intestinal differentiation marker, was high and the expression level of α-SMA, a fibrosis and drug toxicity-related marker, was low in the intestinal organoids of the Normal group, but the expression level of α-SMA increased in the NASH-induced No Treatment group and the OCA- and Eze-treated groups. Meanwhile, it was confirmed that the expression level of the differentiation marker, MUC2, was recovered to some extent in the OCA- and Eze-treated groups compared to the No Treatment group. As a result of analysis on F-actin, an actin-filament marker, and NPC1L1, a protein binding to a critical mediator of cholesterol absorption, it was confirmed that the expression level of F-actin was high and the expression level of NPC1L1 was low in the Normal group, but the expression level of NPC1L1 increased in the No Treatment group because cholesterol accumulated in the liver due to inducement of NASH affected the intestinal organoids. This result coincides with the result of analysis of the mouse organoids. Also, it was confirmed that the expression level of NPC1L1 did not decrease in the OCA-treated group, but decreased in the intestinal organoids of the Eze-treated group. It can be seen that Eze acts as a cholesterol absorption inhibitor and shows the effect of inhibiting cholesterol absorption even in the intestinal organoids.

As a result of optical microscopy and immunofluorescence staining analysis of the human iPSC-derived cardiac organoids in each group, it was confirmed as shown in FIG. 23B that no significant difference among the groups was observed from optical microscopy images, but immunofluorescence staining showed differences among the groups. It was confirmed that the expression levels of CTNT and F-actin, myocardial differentiation and actin-filament markers, were the highest in the cardiac organoids of the Normal group and the lowest in the No Treatment. Also, it was confirmed that the expression of the marker was recovered to some extent in the OCA- and Eze-treated groups. Further, it was confirmed the α-SMA, a fibrosis and drug toxicity-related marker, was hardly expressed in the cardiac organoids of the Normal group, but the expression level of α-SMA was the highest in the No treatment group and α-SMA was expressed in some of the cardiac organoids of the OCA- and Eze-treated groups.

These results demonstrate that even when the multi-organ non-alcoholic steatohepatitis organoid model developed in the present disclosure is fabricated using human iPSC-derived organoids, it can mimic the effect of drug treatment on other organs in an actual NASH patient in vitro, and can serve as a disease model platform that reflects fluidal interactions between organs.

The present disclosure has been described with reference to the preferred exemplary embodiments thereof. It can be understood by a person with ordinary skill in the art that the present disclosure can be implemented as being modified and changed within the scope departing from the spirit and the scope of the present disclosure. Accordingly, the above-described exemplary embodiments should be considered in descriptive sense only and not for purposes of limitation. Also, the technical scope of the present disclosure is defined not by the detailed description of the invention but by the appended claims, and all differences within the scope will be construed as being comprised in the present disclosure.

We claim:

1. A multi-organ model, comprising:
a liver organoid well; and
an intestinal organoid well, a pancreas organoid well, and a cardiac organoid well, each of which is directly or indirectly connected to the liver organoid well by microchannels.

2. The multi-organ model of claim 1,
wherein the intestinal organoid well, the pancreas organoid well, and the cardiac organoid well are not directly connected to each other.

3. The multi-organ model of claim 1,
wherein the microchannels have a cross-sectional width of 10 μm to 30 μm and a height of 5 μm to 20 μm.

4. The multi-organ model of claim 1,
wherein the liver organoid well includes:
a hydrogel containing decellularized liver tissue-derived extracellular matrix (Liver Extracellular Matrix; LEM); and
liver organoids.

5. The multi-organ model of claim 1,
wherein the intestinal organoid well includes a hydrogel containing decellularized intestinal tissue-derived extracellular matrix and intestinal organoids,
the pancreas organoid well includes a hydrogel containing decellularized pancreas tissue-derived extracellular matrix and pancreas organoids, and
the cardiac organoid well includes a hydrogel containing decellularized heart tissue-derived extracellular matrix and cardiac organoids.

6. The multi-organ model of claim 4,
wherein the liver organoids are derived from mouse tissue, human induced pluripotent stem cells (hiPSC) or human liver tissue.

7. A non-alcoholic fatty liver multi-organ model in which the liver organoid well of the multi-organ model of claim 1 is treated with free fatty acid.

8. The non-alcoholic fatty liver multi-organ model of claim 7,
wherein the free fatty acid has a concentration ranging from 100 μM to 900 μM.

9. A method of fabricating a non-alcoholic fatty liver multi-organ model, comprising:
fabricating the multi-organ model of claim 1; and
injecting culture medium containing free fatty acid into the liver organoid well.

10. A method of screening a therapeutic drug for non-alcoholic fatty liver disease, comprising:
treating the non-alcoholic fatty liver multi-organ model of claim 7 with a candidate substance; and
comparing a group treated with the candidate substance to a control group.

11. A method of providing information about drug metabolism of a non-alcoholic fatty liver therapeutic drug on peripheral organs, comprising:
treating the non-alcoholic fatty liver multi-organ model of claim 7 with a candidate substance; and
comparing a group treated with the candidate substance to a control group.

12. A method of evaluating drug toxicity of a non-alcoholic fatty liver therapeutic drug, comprising:
treating the non-alcoholic fatty liver multi-organ model of claim 7 with a candidate substance; and
comparing a group treated with the candidate substance to a control group.
